SEQUENCE LISTING (10 sequences)

SEQ ID NO: 1 - DNA, 1647 bp, Homo sapiens
  CDS (264)..(1382)
ctctccgctgctgtcgccaggagtcacttcacgagaagccaggtcacaaccgtcggcccttgtctggaaaagtaaaagtggatcctgccacgttcggagctccctggcgcctcgcccggctggagctagagaactcgtcctgtggcggcccccggcgtggggcgggacagcggccccctggagggggcagtcccgggagaacctgcggcggccggagcggtaaaaataagtgactaaagaagcagacctgggaatcacctaacatgtcgaggaggagatttgattgccgaagtatttcaggcctactaactacaactcctcaaattccaataaaaatggaaaactttaataatttctatatacttacatctaaagagctagggagaggtaaatttgctgtggttagacaatgtatatcaaaatctactggccaagaatatgctgcaaaatttctaaaaaagagaagaagaggacaggattgtcgggcagaaattttacacgagattgctgtgcttgaattggcaaagtcttgtccccgtgttattaatcttcatgaggtctatgaaaatacaagtgaaatcattttgatattggaatatgctgcaggtggagaaattttcagcctgtgtttacctgagttggctgaaatggtttctgaaaatgatgttatcagactcattaaacaaatacttgaaggagtttattatctacatcagaataacattgtacaccttgatttaaagccacagaatatattactgagcagcatataccctctcggggacattaaaatagtagattttggaatgtctcgaaaaatagggcatgcgtgtgaacttcgggaaatcatgggaacaccagaatatttagctccagaaatcctgaactatgatcccattaccacagcaacagatatgtggaatattggtataatagcatatatgttgttaactcacacatcaccatttgtgggagaagataatcaagaaacatacctcaatatctctcaagttaatgtagattattcggaagaaactttttcatcagtttcacagctggccacagactttattcagagccttttagtaaaaaatccagagaaaagaccaacagcagagatatgcctttctcattcttggctacagcagtgggactttgaaaacttgtttcaccctgaagaaacttccagttcctctcaaactcaggatcattctgtaaggtcctctgaagacaagacttctaaatcctcctgtaatggaacctgtggtgatagagaagacaaagagaatatcccagaggatagcagcatggtttccaaaagatttcgtttcgatgactcattacccaatccccatgaacttgtttcagatttgctctgttagcacttttttctttgactcatttggactgaatttgaaattttatatccactccagtgagattatgatttgtagcttcatatatgacatgtttatattgtaaatgcacttttccatggaataatttagggaagtgttttaatgttaaattactagttgctagcatgttatgatttcatatcctgagatagctctgcagataagaaaatatttaaatatatgacaaaaagtaaaattgtacatgtgagtttacatgttaatgaaataa

SEQ ID NO: 2 - protein, 372 aa, Homo sapiens
MSRRRFDCRSISGLLTTTPQIPIKMENFNNFYILTSKELGRGKFAVVRQCISKSTGQEYAAKFLKKRRRGQDCRAEILHEIAVLELAKSCPRVINLHEVYENTSEIILILEYAAGGEIFSLCLPELAEMVSENDVIRLIKQILEGVYYLHQNNIVHLDLKPQNILLSSIYPLGDIKIVDFGMSRKIGHACELREIMGTPEYLAPEILNYDPITTATDMWNIGIIAYMLLTHTSPFVGEDNQETYLNISQVNVDYSEETFSSVSQLATDFIQSLLVKNPEKRPTAEICLSHSWLQQWDFENLFHPEETSSSSQTQDHSVRSSEDKTSKSSCNGTCGDREDKENIPEDSSMVSKRFRFDDSLPNPHELVSDLLC

SEQ ID NO: 3 - DNA, 2287 bp, Homo sapiens
  CDS (80)..(2287)
aagagccagggaccccaggacccgggaggcggcgcagccggggccgccggaggagcgcgggtgacctggcggcggcgagatgccgctcgcccagctcaaggagccctggccgctcatggagctagtgccgctggacccggagaatggacagacctcaggggaagaagctggacttcagccgtccaaggatgagggcgtcctcaaggagatctccatcacgcaccacgtcaaggctggctctgagaaggctgatccatcccatttcgagctcctcaaggttctgggccagggatcctttggcaaagtcttcctggtgcggaaagtcacccggcctgacagtgggcacctgtatgctatgaaggtgctgaagaaggcaacgctgaaagtacgtgaccgcgtccggaccaagatggagagagacatcctggctgatgtaaatcacccattcgtggtgaagctgcactatgccttccagaccgagggcaagctctatctcattctggacttcctgcgtggtggggacctcttcacccggctctcaaaagaggtgatgttcacggaggaggatgtgaagttttacctggccgagctggctctgggcctggatcacctgcacagcctgggtatcatttacagagacctcaagcctgagaacatccttctggatgaggagggccacatcaaactcactgactttggcctgagcaaagaggccattgaccacgagaagaaggcctattctttctgcgggacagtggagtacatggcccctgaggtcgtcaaccgccagggccactcccatagtgcggactggtggtcctatggggtgttgatgtttgagatgctgacgggctccctgcccttccaggggaaggaccggaaggagaccatgacactgattctgaaggcgaagctaggcatgccccagtttctgagcactgaagcccagagcctcttgcgggccctgttcaagcggaatcctgccaaccggctcggctccggccctgatggggcagaggaaatcaagcggcatgtcttctactccaccattgactggaataagctataccgtcgtgagatcaagccacccttcaagccagcagtggctcagcctgatgacaccttctactttgacaccgagttcacgtcccgcacacccaaggattccccaggcatcccccccagcgctggggcccatcagctgttccggggcttcagcttcgtggccaccggcctgatggaagacgacggcaagcctcgtgccccgcaggcacccctgcactcggtggtacagcaactccatgggaagaacctggtttttagtgacggctacgtggtaaaggagacaattggtgtgggctcctactctgagtgcaagcgctgtgtccacaaggccaccaacatggagtatgctgtcaaggtcattgataagagcaagcgggatccttcagaagagattgagattcttctgcggtacggccagcaccccaacatcatcactctgaaagatgtgtatgatgatggcaaacacgtgtacctggtgacagagctgatgcggggtggggagctgctggacaagatcctgcggcagaagttcttctcagagcgggaggccagctttgtcctgcacaccattggcaaaactgtggagtatctgcactcacagggggttgtgcacagggacctgaagcccagcaacatcctgtatgtggacgagtccgggaatcccgagtgcctgcgcatctgtgactttggttttgccaaacagctgcgggctgagaatgggctcctcatgacaccttgctacacagccaactttgtggcgcctgaggtgctgaagcgccagggctacgatgaaggctgcgacatctggagcctgggcattctgctgtacaccatgctggcaggatatactccatttgccaacggtcccagtgacacaccagaggaaatcctaacccggatcggcagtgggaagtttaccctcagtgggggaaattggaacacagtttcagagacagccaaggacctggtgtccaagatgctacacgtggatccccaccagcgcctcacagctaagcaggttctgcagcatccatgggtcacccagaaagacaagcttccccaaagccagctgtcccaccaggacctacagcttgtgaagggagccatggctgccacgtactccgcactcaacagctccaagcccaccccccagctgaagcccatcgagtcatccatcctggcccagcggcgagtgaggaagttgccatccaccaccctgtga

SEQ ID NO: 4 - protein, 735 aa, Homo sapiens
MPLAQLKEPWPLMELVPLDPENGQTSGEEAGLQPSKDEGVLKEISITHHVKAGSEKADPSHFELLKVLGQGSFGKVFLVRKVTRPDSGHLYAMKVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHSHSADWWSYGVLMFEMLTGSLPFQGKDRKETMTLILKAKLGMPQFLSTEAQSLLRALFKRNPANRLGSGPDGAEEIKRHVFYSTIDWNKLYRREIKPPFKPAVAQPDDTFYFDTEFTSRTPKDSPGIPPSAGAHQLFRGFSFVATGLMEDDGKPRAPQAPLHSVVQQLHGKNLVFSDGYVVKETIGVGSYSECKRCVHKATNMEYAVKVIDKSKRDPSEEIEILLRYGQHPNIITLKDVYDDGKHVYLVTELMRGGELLDKILRQKFFSEREASFVLHTIGKTVEYLHSQGVVHRDLKPSNILYVDESGNPECLRICDFGFAKQLRAENGLLMTPCYTANFVAPEVLKRQGYDEGCDIWSLGILLYTMLAGYTPFANGPSDTPEEILTRIGSGKFTLSGGNWNTVSETAKDLVSKMLHVDPHQRLTAKQVLQHPWVTQKDKLPQSQLSHQDLQLVKGAMAATYSALNSSKPTPQLKPIESSILAQRRVRKLPSTTL

SEQ ID NO: 5 - DNA, 21 bp, Artificial Sequence
  Synthetic construct
gcugccaucuucagggaugtt

SEQ ID NO: 6 - DNA, 21 bp, Artificial Sequence
  Artificial construct
gcugccaucuucagggaugtt

SEQ ID NO: 7 - DNA, 21 bp, Artificial Sequence
  Synthetic construct
uaacauuguucaccuugautt

SEQ ID NO: 8 - DNA, 21 bp, Artificial Sequence
  Synthetic construct
aucaaggugaacaauguuatt

SEQ ID NO: 9 - DNA, 21 bp, Artificial Sequence
  Synthetic construct
cccuaaguguaggacgcactt

SEQ ID NO: 10 - DNA, 21 bp, Artificial Sequence
  Synthetic construct
gugcguccuacacuuagggtt

(19) United States
(12) Patent Application Publication
(10) Pub. No.: US 2010/0183597 A1
(43) Pub. Date: Jul. 22, 2010

(54) DRAK2 EXPRESSION IS ASSOCIATED WITH DIABETES

(76) Inventors: Jiangping Wu, Brossard (CA); Jianning Mao, Montreal (CA)

Correspondence Address: GOUDREAU GAGE DUBUC, 2000 MCGILL COLLEGE, SUITE 2200, MONTREAL, QC, H3A 3H3, CA

(21) Appl. No.: 12/602,893

(22) PCT Filed: Jun. 6, 2008
(86) PCT No.: PCT/CA08/01098

§ 371 Date: Dec. 3, 2009

Related U.S. Application Data
(60) Provisional application No. 60/924,956, filed on Jun. 6, 2007.

(51) Int. Cl. A61K 39/395; A61K 31/7088; A61K 38/16; A61K 38/02; A61K 31/7105; A61P 3/10; G01N 33/567

(52) U.S. Cl. 424/133.1; 514/44 R; 514/12; 514/2; 514/44 A; 435/7.21

(57) ABSTRACT

Drak2 is a member of the death-associated protein family and a serine threonine kinase. In this study, we investigated its role in beta-cell survival and diabetes. Drak2 mRNA and protein were rapidly induced in islet beta-cells after stimulation by inflammatory cytokines known to be present in type 1 diabetes. Drak2 upregulation was accompanied by increased beta-cell apoptosis, beta-cell apoptosis caused by the said stimuli was inhibited by Drak2 knockdown using siRNA. Conversely, transgenic (Tg) Drak2 overexpression led to aggravated beta-cell apoptosis triggered by the stimuli. Further in vivo experiments demonstrated that Drak2 overexpressed in Tg islets is responsible for type 1 diabetes-prone phenotype. Purified Drak2 could phosphorylate ribosomal protein S6 (p70S6) kinase in an in vitro kinase assay. Drak2 overexpression in NIT-1 cells led to enhanced p70S6 kinase phosphorylation, while Drak2 knockdown in these cells reduced it. These mechanistic studies proved that p70S6 kinase was a bona fide Drak2 substrate in vitro and in vivo.

### FIELD OF THE INVENTION

The present invention relates to diabetes and more particularly to islet apoptosis-associated with the disease. More specifically, the present invention is concerned with the survival of islets and with the modulation of apoptosis therein. The present invention thus generally relates to methods for the modulation of islet apoptosis. More particularly, the invention relates to the identification of a kinase whose expression modulates islet apoptosis. The present invention also relates to the identification of a substrate of that kinase and of its involvement in apoptosis modulation in diabetes. The present invention therefore relates to the identification of a pathway, which can be targeted to modulate islet apoptosis. In general, the present invention thus relates to diabetes diagnosis, treatment and monitoring by methods and/or compounds that modulate or monitor expression of the identified kinase and substrate thereof. Additionally, the invention relates to screening assays to identify modulators of the kinase of the invention expression or activity.

### BACKGROUND OF THE INVENTION

Diabetes is a metabolic disorder in which the pancreatic islets fail to produce sufficient insulin to prevent blood glucose from rising beyond a normal range. Type I diabetes (T1D) is an autoimmune disease normally starting at a young age. In T1D, insufficient insulin production is caused by the destruction of islets by T cells either directly or indirectly by inflammatory cytokines such as IFNβ and/or TNFβ plus IL-R (Hohmeier et al., 2003. Int. J. Obes. Relat. Metab. Disord. 27 Suppl 3:S12-S16). Increased blood glucose and lipid levels after the onset of T1D in turn aggravate islet destruction, due to glucolipotoxicity (Wilkin 2001. Diabetologia 44: 914-922). Due to calorie-rich diet and sedative life-style, obesity is epidemic in industrialized countries. Taking the US as an example, 30% of its population are obese and 50% are overweight (Wild et al., 2004. Diabetes Care 27:1047-1053.) Obesity favours the development of the metabolic syndrome, of which type 2 diabetes (T2D) is one manifestation. T2D thus has a later onset in life. In T2D, reduced insulin sensitivity is the major problem (Lockwood et al., 1983. Am. J. Med. 75:23-31). However, recent research has revealed that adipose and other tissues in T2D release harmful inflammatory cytokines, which are detrimental to islet function and survival (Kahn et al., 2006. Nature 444: 840-846). In the late stage of T2D, as it is the case in TD1, increased blood glucose and lipid contribute to islet destruction because of glucolipotoxicity (Wilkin, 2001. Supra. Science 307:380-384). Thus, T1D and T2D appear to represent two extremes of a spectrum, with different degrees and tempo of islet destruction caused by inflammation and glucolipotoxicity. It is conceivable that genes controlling islet apoptosis and survival are important in determining susceptibility to islet destruction, and, consequently, diabetes risk as well as its onset tempo (Chacon et al., 2007. Atherosclerosis volume. Such genes can, therefore, be characterized as diabetes risk genes for both T1D and T2D and thus, their identification would be valuable to diagnose, treat and/or monitor onset and/or progression of both types of the diabetes.

Drak2 is a serine/threonine kinase belonging to a family of death-associated protein kinases (DAP kinases). The DAP kinase family comprises DAP (Deiss et al., 1995. Genes Dev. 9:15-30.), DRP-1 (Inbal et al., 2000. Mol. Cell. Biol. 20:1044-1054), ZIP kinase (Kawai, T. et al., 1998. Mol. Cell. Biol. 18:1642-1651), DAPK2 (Kawai, T et al., 1999. Oncogene 18:3471-3480), and Drak1 and Drak2 (Sanjo, et al., 1998. J. Biol. Chem. 273:29066-29071). Drak2 shares about 50% identity in the kinase domain with other members of the family (Deiss et al., 1995. Genes Dev. 9:15-30.). While DAP, DRP-1 and DAPK2 have a calmodulin regulatory domain in their C-terminal, ZIP, Drak1 and Drak2 do not (Deiss et al., 1995. Supra; Inbal et al., 2000 Supra; Kawai et al., 1998 and 1999 Supra; Sanjo et al., 1998, Supra). DAP, DAPK2, and DRP-1 are localized in the cytosol (Deiss et al., 1995, Supra); Inbal et al., 2000, Supra; Kawai et al., 1999, Supra) whereas ZIP kinase and Drak1 reside mainly in the nuclei (Kawai et al., 1998, Supra; Sanjo et al., 1998, Supra) and Drak2 is found in both the cytosol and nuclei (Sanjo et al., 1998, Supra); Matsumoto et al., 2001; J. Biochem. (Tokyo) 130:217-225), suggesting different mechanisms of action. Drak2 autophosphorylates itself, and phosphorylates myosin light chain as an exogenous substrate (Sanjo et al., 1998, Supra). Its endogenous substrates, other than itself, have not been identified. Drak2 interacts with a calcineurin homologous protein (Matsumoto et al., 2001, Supra) but the biological significance of this interaction is not clear. In any event, there remains a need to identify and characterize other substrates of Drak2.

According to DNA microarray (Su et al., 2002. Proc., Natl. Acad. Sci. U.S.A 99: 4465-4470) and real-time reverse transcription-polymerase chain reaction (RT-PCR) analysis (McGargill et al., 2004. Immunity. 21:781-791) of different tissues, Drak2 was reported to be exclusively expressed in the T-cell compartment. However, in situ hybridization analysis revealed that Drak2 expression is ubiquitous at the mid-gestation stage in embryos, followed by more focal expression in various organs in the perinatal period and adulthood, notably in the thymus, spleen, lymph nodes, cerebellum, suprachiasmatic nuclei, pituitary, olfactory lobes, adrenal medulla, stomach, skin and testes (Mao et al., 2006. J. Biol. Chem. 281: 12587-12595). Such an expression pattern suggests that Drak2 has a more fundamental function in cell biology.

When DAP family kinases are overexpressed in various cells, apoptosis ensues (Deiss et al., 1995. Supra; Inbal et al., 2000. Supra; Kawai et al., 1998. Supra; Kawai et al., 1999. Supra; Sanjo et al., 1998. Supra) indicating their involvement in apoptosis. The immune system of Drak2 null-mutant mice has been investigated by McGargill et al., 2004 (Supra) and Wu et al., (Wu et al., 2004. Transplantation 78:360-366.). In vitro, Drak2^{-/-} T cells have no apparent defect in activation-induced apoptosis, after stimulation with anti-CD3 and anti-CD28; this lead to the conclusion that Drak2 did not play a significant role in T-cell apoptosis. However, in Drak2 transgenic (Tg) mice, Tg T cells manifest augmented apoptosis after TCR stimulation followed by culture in the presence of IL-2. As a consequence, the memory T-cell pool is diminished, and the Tg mice incur compromised secondary but not primary in vivo T-cell responses (Mao et al., 2006, Supra). These results therefore reveal that Drak2 is important in regulating T-cell apoptosis both in vitro and in vivo.

There thus remains a need for novel methods of modulating apoptosis-associated with TD1 and TD2.

There also remains a need for identifying new therapeutic targets allowing the modulation of apoptosis associated with diabetes.

In addition, there remains a need to develop new therapeutic strategies for the treatment of diabetes.

The present invention seeks to meet these and other needs.

The present description refers to a number of documents, the content of which is herein incorporated by reference in their entirety.

### SUMMARY OF THE INVENTION

The present inventions relates to the identification of a kinase pathway leading to and stemming from the Drak2 death associated kinase, as a pathway involved in the modulation of apoptosis of islet cells.

The present invention thus relates to the identification of Drak2 as a gene target for diabetes diagnosis, treatment (e.g., treatment prediction, and treatment response), and studies. More particularly, the invention teaches that a decrease in expression/activity of Drak2 protects islets from apoptosis.

The present invention further relates to the identification of the S6 kinase as a substrate of Drak2 kinase.

Further, the instant invention relates to a method for decreasing expression/activity of Drak2 and for decreasing expression/activity of S6 thereby further protecting islets from apoptosis (e.g., and the use of a composition comprising agents which decrease expression/activity of Drak2 and S6).

Furthermore, the instant invention relates to a method for decreasing expression/activity of Drak2 and for decreasing expression/activity of S6 together with a further decreasing of the level/activity of cytokines (e.g., TGF, IL-1, IFN) involved in islet apoptosis thereby further protecting islets from apoptosis (e.g., and the use of a composition comprising agents which decrease expression/activity of Drak2 and S6 and lower the level and/or activity of cytokines involved in islet apoptosis).

In the course of our search for genes affecting islet survival, it was discovered that Drak2 expression in islets was rapidly induced by free fatty acids (FFA). It was also discovered that Drak2 expression in islets was rapidly induced by inflammatory stimuli and that the induction was accompanied by islet apoptosis. Truncation of such Drak2 upregulation protected β-cells from apoptosis thus induced. Conversely, Drak2 overexpression in transgenic (Tg) islets resulted in increased β-cell death in vitro upon FFA stimulation, and Drak2 Tg mice developed glucose intolerance after diet-induced obesity. Thus, Drak2 Tg mice were prone to T1D and T2D in vivo.

In addition, it is thus further shown herein that ribosomal protein S6 p70S6 kinase is a substrate of Drak2.

Herein, it is thus demonstrated that Drak2 is critical in β-cell apoptosis triggered by inflammatory cytokines and FFA. Further in vivo experiments proved that enhanced Drak2 expression increased both T1D and T2D risks. Drak2 would thus be in a common pathway leading to harmful signals received by islets in T1D and T2D environments.

The present invention has confirmed that Drak2 is not a gene which expression is restricted to the T-cell compartment. It also showed that contrarily to what was suggested initially (McGargill et al., 2004, Supra) Drak2 does play an essential role in apoptosis. It is shown herein that it is not only upregulated in islet β-cells upon stimulation, but that it is also pivotal in islet cells function and survival, which are compromised in both T1D and T2D. This thus supports the notion that T1D and T2D represent the 2 extremes of a spectrum, and Drak2 is one of the common denominators. As a consequence, based on the herein presented results with the animal model, Drak2 can be considered a risk factor for both T1D and T2D. Without being limited to a particular theory it can be hypothesized: that subpathogenic levels of inflammatory cytokines or FFA for normal individuals, culminate in islet death in patients with abnormally high Drak2 level activities; chronic accumulation of such islet deaths eventually leads to overt diabetes.

Prior to the present invention, the knowledge about the Drak2 activation pathway and Drak2 substrates was limited, since it was only known that Drak2 is a genuine substrate of itself.

We have now identified 5 putative Drak2 substrates, and proven that p70S6 kinase was a bona fide Drak2 substrate both in vitro and in vivo. While the verification of the other 4 substrates is ongoing, it nevertheless appears that Drak2 has multiple substrates.

As shown herein, when Drak2 upregulation stimulated by cytokines or FFA was truncated by an inhibitor such as siRNA, while islet apoptosis was reduced, it was not totally prevented. As siRNA inhibition of Drak2 expression is not total, the following 2 possibilities were indistinguishable: a) residual Drak2 activity in the siRNA-transfected cells contributed to the remaining apoptosis, or 2) Drak2 is only one of several apoptosis pathways involved in cytokine- or FFA-stimulated islet death.

The present invention having identified Drak2 as a potential diabetes risk factor common to both T1D and T2D. Drak2 is therefore a valid drug target for preventing or delaying the onset of T1D and T2D. Therefore, the present invention also relates to a method for diagnosing a risk of developing diabetes (either type1 or type2 diabetes) in a susceptible subject, which comprises the step of measuring a level or activity of Drak2 in said susceptible subject's tissue or cells which is higher than that in a control subject, as an indication of a risk of developing diabetes.

It is further another object of this invention to provide a method for preventing or delaying the onset of diabetes (either type1 or type2 diabetes) in a susceptible subject, which comprises the step of inhibiting the increase of Drak2 level/activity. In an another embodiment, the method for preventing or delaying the onset of T1D or T2D in a susceptible subject, comprises the step of inhibiting the increase of Drak2 level/activity and of S6K level/activity.

Drak2 is upregulated in islet β-cells upon FFA stimulation, and such upregulation is correlated to decreased islet function and survival. Interestingly, although Tg islets had higher Drak2 expression, such over expression by itself did not manifest harmful effects on the islets, as Tg mice did not develop diabetes. Furthermore, Tg islets culture in medium did not suffer from increased apoptosis, as compared to wild-type (WT) islets, until an exogenous detrimental factor (e.g., FFA) was present. Again, without being limited to a particular theory, this suggests that Drak2 might act on a two-hit mode, in which other signaling events (hit 1) derived from FFA stimulation as well as Drak2 (hit 2) are both required to trigger 11-cell damage and/or dysfunction. For normal islets, high Drak2 expression (hit 2) could be a consequence of FFA (hit 1). It can be hypothesized that in individuals with abnormally high basal Drak2 expression levels in islets, a lesser hit 1 might be sufficient to cause excessive islet damage or dysfunction. Such individuals would be more prone to T2D development when facing increased serum lipid. Of interest, in humans, Drak2 gene is located in 2q33.2, and is 14.8 Mbp away from a type 2 diabetes risk region at 2q32.1 (http://www.ncbi.nlm.nih.gov/entrez/dispomim.cgi?cmd=entry&id=601724).

Further studies shown herein reinforce the conclusion that Drak2 is critical for β-cell apoptosis triggered by inflammatory cytokines. Further, additional in vivo experiments proved that enhanced Drak2 expression in islets rendered mice prone to type 1 diabetes. In addition, p70S6 kinase was identified as a Drak2 substrate. Drak2 is highly conserved among species (see below).

According to the present invention, Tg islets manifested compromised function after cytokine assaults. Indeed, without such assaults, insulin release of Tg islets was not different from that of WT islets. Thus, the present studies suggest that Drak2 overexpression, by itself, is not sufficient to cause β-cell dysfunction and apoptosis. Rather, Drak2 overexpression renders β-cells vulnerable to signaling from other detrimental factors. Indeed, islet β-cell apoptosis often needs concerted signals from different pathways. For example, a single cytokine such as TNF, IFN or IL-1R does not have a significant effect on β-cells, but a combination of 2 or 3 of them potently induces their apoptosis (Cnop, et al., 2005. Diabetes 54 Suppl 2, S97-S107). The present findings are also consistent with the fact that T1D is under polygenic control, and that abnormal expression of a single gene rarely induces diabetes. Thus, the present invention also relates to apoptosis protection by also targeting at least one cytokine.

In humans, the Drak2 gene is located in 2q33.2, and is 7.2 Mbp from a type 1 diabetes risk locus IDDM12 at 2q33.2. Although CTLA-4 has been identified in this locus (Turpeinen et al., 2003. Eur. J. Immunogenet. 30:289-293), whether there are additional type 1 diabetes risk genes in this area needs to be assessed.

p70S6 kinase plays a critical role in protein synthesis, and is a key regulator in cell size and cell cycle progression. Accordingly, its sequence has been conserved troughout evolution (see below). It is activated through phosphorylation triggered by a wide range of growth factors, cytokines and nutrients (Jastrzebski et al., 2007. Growth Factors 25:209-226). mTORC1 and PDK1 are 2 known kinases which work in concert to phosphorylate and activate p70S6 kinase. Herein, we have identified a novel p70S6 kinase signalling pathway in which Drak2 is an additional upstream kinase capable of phosphorylating p70S6 kinase.

Thus, the present invention shows that the inflammatory cytokine/Drak2/p70S6 kinase pathway is critical in islet apoptosis, because the action of all these 3 components was correlated to islet apoptosis, and they were sequentially linked. Inhibitors of components of this pathway should have protective effects on β-cells.

Interestingly, islet transplantation efficiency has been greatly improved after rapamycin (also known commercially as sirolimus), a mTORC1 inhibitor, replaced the calcineurin inhibitor cyclosporin A in the islet transplantation regimen (Marcelli-Tourvieille et al., 2007. Transplantation 83, 532-538). It is conceivable that inhibition of p70S6 kinase phosphorylation by rapamycin contributes to reduce islet apoptosis after transplantation, and hence, is partially responsible for the increase in transplantation efficiency (Marcelli-Tourvieille et al., 2007. Supra).

The present invention, provides in vitro evidence that rapamycin renders β-cells partially resistant to apoptosis. Thus the present invention validates p70S6 kinase as relevant to islet survival. It is possible that inflammatory cytokines activate both the Drak2/p70S6 kinase and mTORC1/p70S6 kinase pathways, and that inhibiting one of them is only partially effective in reducing β-cell apoptosis. Indeed, when Drak2 upregulation stimulated by cytokines was prevented by siRNA, islet apoptosis was decreased, but was not totally prevented. Similarly, rapamycin only partially protected islet apoptosis from the cytokines. Dual inhibition of mTORC1 (with rapamycin) and Drak2 might thus achieve better results in islet protection in terms of cytokine-induced β-cell apoptosis.

Indeed, the present invention also demonstrates that a dual inhibition of the Drak2/p70S6 kinase and mTORC1/p70S6 kinase pathways showed an additive protective effect as compared to an inhibition of only one of the pathways (in both mouse and human models).

In yet another embodiment, the invention relates to a method for increasing the survival of β-cell upon transplantation thereof in a patient in need of such a transplantation, the method comprising the use of an agent which decreases the expression of Drak2, or β-cell expressing a lower level or a less functional Drak2, thereby increasing the survival of the β-cell upon transplantation thereof in the patient in need thereof. In a related embodiment, the cells to be transplanted are also treated so as to have a decrease level or activity of S6.

The present invention is based on the demonstration of the importance of Drak2 in islet cell function and survival, and its identification as a new therapeutic targets for the modulation of apoptosis thereof. Since both T1D and T2D share β-cell apoptosis in disease onset or progression, Drak2 is herein identified a new therapeutic and diagnosis target for diabetes.

As shown herein, overexpression of Drak2 promotes apoptosis of β-cell. Conversely, decrease in Drak2 expression in mouse or human NK cells was found to reduce apoptosis. Further experiments revealed that Drak2 also phosphorylates the S6kinase.

Thus, not only is Drak2 identified as a novel therapeutic target to modulate the apoptosis of islet cells, but a combination of a modulation of the Drak2/S6kinase and mTORC1/S6kinase pathways further modulates the apoptosis pathway in these cells.

Thus, in one aspect, the present invention relates to the inhibition of the expression or functions of Drak2 (alone or together with that of mTORC1/S6kinase pathway) in order to reduce β-cell apoptosis.

In another aspect, the present invention relates to the increase of the expression or functions of Drak2 (alone or together with that of mTORC1/S6kinase pathway) in order to augment β-cell apoptosis.

In one embodiment, the methods of the present invention comprise a modulation of the expression of Drak2 in a cell or organism. Such methods include, in particular embodiments, the use of an antisense nucleic acid of DRAK2, of DRAK2 siRNAs or of a DRAK2 specific ribozyme. Other agents, which decrease the expression level and/or activity of DRAK2 (e.g., nuclear antibodies, small molecules, peptides) are also encompassed as agents useful for reducing islet β-cell apoptosis and to treat or prevent diabetes.

Thus, in a related aspect, the present invention concerns antisense oligonucleotides hybridizing to a nucleic acid sequence encoding DRAK2 protein (SEQ ID NO:2) thereby enabling the control of the transcription or translation of the DRAK2 gene in cells. The antisense sequences of the present invention consist of all or part of the DRAK2 nucleic acid sequence (SEQ ID NO:1, Genbank Accession number BC_{—}016040) in reverse orientation, and variants thereof. The present invention further relates to small double stranded RNA molecules (siRNAs) derived from DRAK2 nucleic acid sequence (SEQ ID NO:1, Genbank Accession number BC_{—}016040) which also decrease DRAK2 protein cell expression. In a particular embodiment, the present invention relates to antisense oligonucleotides and siRNAs that inhibit the expression of DRAK2 and protect against apoptosis. The present invention also relates to methods utilizing siRNA or antisense RNA to reduce DRAK2 mRNA and/or protein expression and therefore, to increase β-cell function or survival which are in part dependent on DRAK2 expression and biological activity. In a particular embodiment, inhibition or reduction of DRAK2 expression significantly protects β-cell. In another embodiment, increase of DRAK2 expression significantly increases apoptosis of β-cell. The DRAK2 complementary sequences of the present invention can either be directly transcribed in target cells or synthetically produced and incorporated into cells by well-known methods.

In a related aspect, the present invention features a method of reducing DRAK2 expression in a subject by administering thereto a RNA, or derivative thereof (e.g., siRNA, antisense RNA, etc), or vector producing same in an effective amount, to reduce DRAK2 expression, thereby increasing β-cell survival or function and treating or preventing a disease such as diabetes. The RNA (e.g., siRNA, antisense RNA, etc) can be modified so as to be less susceptible to enzymatic degradation or to facilitate its delivery to a target cell (e.g., β-cell). RNA interference (i.e., RNAi) toward a targeted DNA segment in a cell can be achieved by administering a double stranded RNA (e.g., siRNA) molecule to the cell, wherein the ribonucleotide sequence of the double stranded RNA molecule corresponds to the ribonucleotide sequence of the targeted DNA segment. In one particular case where the siRNA or antisense RNA is chemically modified or contains point mutations, the antisense region of the siRNAs or antisense RNA, of the present invention is still capable (i.e., of maintaining its ability to hybridize to the target sequence) of hybridizing to the ribonucleotide sequence of the targeted gene (e.g., DRAK2 mRNA) and to inhibit its expression (e.g., trigger RNAi).

In another embodiment, the present invention relates to the use of DRAK2 specific ribozymes to reduce DRAK2 expression in cells and thus to protect β-cell functions or level (e.g., decrease apoptosis of islet cells in diabetes). As well known in the art, ribozymes are enzymatic nucleic acid molecules capable of catalyzing the cleavage of other separate nucleic acid molecules in a nucleotide base sequence-specific manner. They can be used to target virtually any RNA transcript (see for example U.S. Pat. No. 6,656,731). Such event renders the targeted mRNA non-functional and abrogates protein expression of the target RNA. Thus, in accordance with one embodiment of the present invention DRAK2 expression is inhibited by the use of DRAK2 specific ribozymes in order to enhance protection of islet cells.

In a further embodiment, the present invention relates to screening assays to identify compounds that modulate the biological activity of DRAK2.

In one particular aspect, the present invention relates to screening assays to identify compounds (e.g., peptides, nucleic acids, small molecules) that completely or partially inhibit the expression of DRAK2, thereby protecting against apoptosis.

In another aspect, the invention provides assays for screening candidates or test compounds, which bind to or modulate the activity of an DRAK2 protein or polypeptide or biologically active portion thereof. Thus, screening assays to identify compounds which reduce DRAK2 expression or activity are encompassed by the present invention. Such compounds may be useful in the treatment of diabetes and other autoimmune diseases such as lupus and rheumatoid arthritis.

In one embodiment, the assay is a cell-based assay in which a cell which expresses a DRAK2 protein or biologically active portion thereof, either natural or of recombinant origin, is contacted with a test compound and the ability of same to modulate a biological activity of DRAK2, e.g., autologous phosphorylation, interaction with downstream effectors, apoptosis assay, kinasing of S6 or other measurable biological activity of DRAK2, is determined. Determining the ability of same to modulate DRAK2 activity can also be accomplished by monitoring, for example, the expression and/or activity of a specific gene modulated by a DRAK2-dependent signalization cascade in the presence of the test compound as compared to the expression and/or activity in the absence thereof.

In yet a further embodiment, modulators of DRAK2 expression are identified in a method wherein a cell is contacted with a candidate compound and the expression of DRAK2 mRNA or protein in the cell is determined. The level of expression of DRAK2 mRNA or protein in the presence of the candidate compound is compared to the level of expression of DRAK2 mRNA or protein in the absence of the candidate compound. The candidate compound can then be identified as a modulator of DRAK2 expression based on this comparison. For example, when expression of DRAK2 mRNA or protein is greater (statistically significantly greater) in the presence of the candidate compound than in its absence, the candidate compound is identified as a stimulator of DRAK2 mRNA or protein expression. Alternatively, when expression of DRAK2 mRNA or protein is less (statistically significantly less) in the presence of the candidate compound than in its absence, the candidate compound is identified as an inhibitor of DRAK2 mRNA or protein expression. The level of DRAK2 mRNA or protein expression in the cells can be determined by methods described herein or other methods known in the art for detecting DRAK2 mRNA or protein.

In one embodiment, the screening assays of the present invention comprise: 1) contacting an DRAK2 protein, or functional variant thereof, with a candidate compound; and 2) measuring a biological activity of DRAK2, or variant thereof, in the presence of the candidate compound, wherein a compound that inhibits DRAK2 function is selected when a DRAK2 biological activity is significantly reduced in the presence of said candidate compound as compared to in the absence thereof.

The compounds identified by the screening assays of the present invention can be used as competitive or non-competitive inhibitors in assays to screen for, or to characterize similar or new DRAK2 antagonists. In competitive assays, the compounds of the present invention can be used without modification or they can be labelled (i.e., covalently or non-covalently linked to a moiety which directly or indirectly provide a detectable signal). Examples of labels include radiolabels such as 125I, 14C, and 3H, enzymes such as alkaline phosphatase and horseradish peroxidase (U.S. Pat. No. 3,645,090), ligands such as biotin, avidin, and luminescent compounds including bioluminescent, phosphorescent, chemiluminescent and fluorescent labels (U.S. Pat. No. 3,940,475).

In a related aspect, the present invention also relates to the use of any compound capable of inhibiting (antagonist, e.g., compound which reduces the phosphorylation of DRAK2) or stimulating (agonist, e.g., compound which stimulates the phosphorylation of DRAK2) DRAK2 expression in a cell for the preparation of a pharmaceutical composition intended for the for example the treatment or prevention of diabetes.

In a further embodiment, the present invention features pharmaceutical composition comprising a compound of the present invention (e.g., antisense, siRNA, ribozyme, peptides, nucleic acids, small molecules, antibodies etc) which can be chemically modified, in a pharmaceutically acceptable carrier or diluent. In another embodiment, the present invention features a method for treating or preventing a disease or condition in a subject (e.g., viral infections, cancers, autoimmune diseases), comprising administering to the subject a composition of the invention under conditions suitable for the treatment or prevention of the disease or condition in the subject (e.g., viral infections, cancers, autoimmune diseases), alone, or in conjunction with one or more therapeutic compounds.

In one embodiment, pharmaceutical compositions of the present invention comprise a specific nucleic acid sequence (e.g., a mammalian DRAK2 sequence, siRNA, antisense and the like) or fragment thereof in a vector, under the control of appropriate regulatory sequences to target its expression into a cell.

The methods of the present invention can be used for subjects with pre-existing condition (e.g., already suffering from diabetes), or subject predisposed to such condition. Thus, the present invention also relates to a prevention or prophylaxy of a disease or condition using the reagents and methods of the present invention.

The compounds of the present invention include lead compounds and derivative compounds constructed so as to have the same or similar molecular structure or shape, as the lead compounds, but may differ from the lead compounds either with respect to susceptibility to hydrolysis or proteolysis (e.g., bioavailability), or with respect to their biological properties (e.g., increased affinity for DRAK2). The present invention also relates to compounds and compositions that are useful for the treatment or prevention of conditions, diseases or disorders associated with inappropriate DRAK2 production or function.

In another embodiment, the present invention also relates to pharmaceutical compositions comprising one or more of the compounds described herein and a physiologically acceptable carrier. These pharmaceutical compositions can be in a variety of forms including oral dosage forms, topic creams, suppository, nasal spray and inhaler, as well as injectable and infusible solutions. Methods for preparing pharmaceutical composition are well known in the art as reference can be made to Remington's Pharmaceutical Sciences, Mack Publishing Company, Eaton, Pa., USA.

The compounds of the present invention can be administered to a subject to completely or partially inhibit the activity of DRAK2 in vivo. Thus the methods of the present invention are useful in the therapeutic treatment of DRAK2 related diseases which would benefit from an apoptotic inhibitor. For example, the compositions of the present invention can be administered in a therapeutically effective amount to treat symptoms related to inappropriate diabetes. In addition, the compounds of the present invention may be utilized alone or in combination with any other appropriate therapies (e.g., rapamycin, inhibitors of cytokine level/activity), as determined by the practitioner.

In order to provide a clear and consistent understanding of terms used in the specification and claims, including the scope to be given such terms, a number of definitions are provided herein below.

### DEFINITIONS

Unless defined otherwise, the scientific and technological terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), Rieger et al., Glossary of genetics: Classical and molecular, 5th edition, Springer-Verlag, New-York, 1991; Alberts et al., Molecular Biology of the Cell, 4th edition, Garland science, New-York, 2002; and, Lewin, Genes VII, Oxford University Press, New-York, 2000. Generally, the methods traditionally used in molecular biology, such as preparative extractions of plasmid DNA, centrifugation of plasmid DNA in caesium chloride gradient, agarose or acrylamide gel electrophoresis, purification of DNA fragments by electroelution, phenol or phenol-chloroform extraction of proteins, ethanol or isopropanol precipitation of DNA in saline medium, transformation into bacteria or transfection into cells, procedure for cell culture, infection, methods and the like are common methods used in the art. Such standard techniques can be found in reference manuals such as for example Sambrook et al. (2000, Molecular Cloning—A Laboratory Manual, Third Edition, Cold Spring Harbour Laboratories); and Ausubel et al. (1994, Current Protocols in Molecular Biology, John Wiley & Sons, New-York). In addition, methods and procedures to produce transgenic animals are well-known in the art and described in details for example in: Hogan et al., 1994, Manipulating the Mouse Embryo, Cold Spring Harbor Laboratory Press; Nagy et al., 2002, Manipulating the Mouse Embryo, 3rd edition, Cold Spring Harbor Laboratory Press.

The use of the word “a” or “an” when used in conjunction with the term “comprising” in the claims and/or the specification may mean “one” but it is also consistent with the meaning of “one or more”, “at least one”, and “one or more than one”.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term about.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

Nucleotide sequences are presented herein by single strand, in the 5′ to 3′ direction, from left to right, using the one-letter nucleotide symbols as commonly used in the art and in accordance with the recommendations of the IUPAC IUB Biochemical Nomenclature Commission.

As used herein, “nucleic acid molecule” or “polynucleotides”, refers to a polymer of nucleotides. Non-limiting examples thereof include DNA (e.g., genomic DNA, cDNA), RNA molecules (e.g., mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the terms “nucleic acid” and “polynucleotides” as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Int'l Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Intl Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

The terminology “DRAK2 nucleic acid” or “DRAK2 polynucleotide” refers to a native DRAK2 nucleic acid sequence. In one embodiment, the human DRAK2 nucleic acid sequence is as set forth in SeQ ID NO:1). Other sequences are shown in FIG. 16, since the siRNA designed from mouse were effective in humans An “isolated nucleic acid molecule”, as is generally understood and used herein, refers to a polymer of nucleotides, and includes but should not be limited to DNA and RNA. The “isolated” nucleic acid molecule is purified from its natural in vivo state.

By “RNA” or “mRNA” is meant a molecule comprising at least one ribonucleotide residue. By ribonucleotide is meant a nucleotide with a hydroxyl group at the 2′ position of a R-D-ribo-furanose moiety. The term include double stranded RNA, single stranded RNA, isolated RNA such as partially purified RNA, essentially purified RNA, synthetic RNA, recombinantly produced RNA, as well as altered RNA that differs from naturally occurring RNA by the addition, deletion, substitution and/or alteration of one or more nucleotide. Such alterations can include addition of non-nucleotide material, such as to the end(s) of a siRNA or internally, for example at one or more nucleotides of the RNA molecule. Nucleotides in the RNA molecules of the instant invention can also comprise non-standard nucleotides or chemically synthesized nucleotides or deoxynucleotides. These altered RNAs can be referred to as analogs or analogs of naturally occurring RNA.

Complementary DNA (cDNA). Recombinant nucleic acid molecules synthesized by reverse transcription of messenger RNA (“mRNA”).

Expression. By the term “expression” is meant the process by which a gene or otherwise nucleic acid sequence produces a polypeptide. It involves transcription of the gene into mRNA, and the translation of such mRNA into polypeptide(s).

The term “vector” is commonly known in the art and defines a plasmid DNA, phage DNA, viral DNA and the like, which can serve as a DNA vehicle into which nucleic acid of the present invention can be cloned. Numerous types of vectors exist and are well known in the art. One specific type of vector is called a targeting vector which may be used for homologous recombination with an endogenous target gene in a cell. Homologous recombination occurs between two sequences (i.e. the targeting vector and endogenous gene sequences) that are partially or fully complementary. Homologous recombination may be used to alter a gene sequence in a cell (e.g., embryonic stem cells, (ES cells)) in order to completely shut down protein expression or to introduce point mutations, substitutions or deletions in the target gene sequence. Such method is used for example to generate transgenic animals and is well known in the art.

Expression Vector. A vector or vehicle similar to a cloning vector but which is capable of expressing a gene which has been cloned into it, after transformation into a host. The cloned gene (or nucleic acid sequence) is usually placed under the control of (i.e., operably linked to) certain control sequences such as promoter sequences which may be cell or tissue specific (e.g., pancreas).

Expression control sequences will vary depending on whether the vector is designed to express the operably linked gene (or nucleic acid sequence) in a prokaryotic and/or eukaryotic host and can additionally contain transcriptional elements such as enhancer elements, termination sequences, tissue-specificity elements, and/or translational initiation and termination sites. Vectors which can be used both in prokaryotic and eukaryotic cells are often called shuttle vectors. In particular embodiment, the control sequences may allow general expression (i.e. expression in a large number of cell types) or tissue specific or cell specific expression of a particular nucleic acid sequence.

A DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain Shine Dalgarno sequences in addition to the −10 and −35 consensus sequences.

As used herein, the term “gene therapy” relates to the introduction and expression in an animal (preferably a human) of an exogenous sequence (e.g., a DRAK2 or preferably non-functional Drak2 (in terms of promoting apoptosis), a DRAK2 siRNA or antisense nucleic acid) to supplement, replace or inhibit a target gene (i.e., DRAK2gene), or to enable target cells to produce a protein (e.g., a DRAK2 chimeric protein to target a specific molecule or compete out a binding agent of WT Drak2). In a particular embodiment, the exogenous sequence is of the same origin as that of the animal (human sequence). In another embodiment, the exogenous sequence is of a different origin (e.g., human exogenous sequence in mice (e.g., knock-in).

Nucleic acid sequences may be detected by using hybridization with a complementary sequence (e.g., oligonucleotide probes—see U.S. Pat. Nos. 5,503,980 (Cantor); 5,202,231 (Drmanac et al.); 5,149,625 (Church et al.); 5,112,736 (Caldwell et al.); 5,068,176 (Vijg et al.); and 5,002,867 (Macevicz)). Hybridization detection methods may use an array of probes (e.g., on a DNA chip) to provide sequence information about the target nucleic acid which selectively hybridizes to an exactly complementary probe sequence in a set of four related probe sequences that differ by one nucleotide (see U.S. Pat. Nos. 5,837,832 and 5,861,242 (Chee et al.). In addition, any other well-known hybridization technique (Northern blot, dot blot, Southern blot) may be used in accordance with the present invention.

Nucleic Acid Hybridization. Nucleic acid hybridization depends on the principle that two single-stranded nucleic acid molecules that have complementary base sequences will reform the thermodynamically favoured double-stranded structure if they are mixed under the proper conditions. The double-stranded structure will be formed between two complementary single-stranded nucleic acids even if one is immobilized on a nitrocellulose filter. In the Southern or Northern hybridization procedures, the latter situation occurs. The DNA/RNA of the individual to be tested may be digested with a restriction endonuclease if applicable, prior to its fractionation by agarose gel electrophoresis, conversion to the single-stranded form, and transfer to nitrocellulose paper, making it available for reannealing to the hybridization probe. Non-limiting examples of hybridization conditions can be found in Ausubel, F. M. et al., Current protocols in Molecular Biology, John Wiley & Sons, Inc., New York, N.Y. (1994). For purposes of illustration, an example of moderately stringent conditions for testing the hybridization of a polynucleotide of the present invention with other polynucleotides includes prewashing in a solution of 5×SSC, 0.5% SDS, 1 mM EDTA (pH 8.0); hybridizing at 50° C.-60° C., 5×SSC and 100 μg/ml denatured salmon sperm DNA overnight (12-16 hours); followed by washing twice at 60° C. for 15 minutes with each of 2×SSC, 0.5×SSC and 0.2×SSC containing 0.1% SDS. For example for highly stringent hybridization conditions, the hybridization temperature is changed to 62, 63, 64, 65, 66, 67 or 68° C. One skilled in the art will understand that the stringency of hybridization can be readily manipulated, such as by altering the salt and SDS concentration of the hybridizing and washing solutions and/or temperature at which the hybridization is performed. The temperature and salt concentration selected is determined based on the melting temperature (Tm) of the DNA hybrid. Other protocols or commercially available hybridization kits using different annealing and washing solutions can also be used as well known in the art. The use of formamide in different mixtures to lower the melting temperature may also be used and is well known in the art.

A “probe” is meant to include a nucleic acid oligomer that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.”

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) non standard base pairing (e.g., I:C) or may contain one or more residues (including a basic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. In reference to more specific nucleic acid molecules of the present invention, the binding free energy for a nucleic acid molecule with its complementary sequence is sufficient to allow the relevant function of the nucleic acid to proceed (e.g., RNAi activity). For example, the degree of complementarity between the sense and antisense region (or strand) of the siRNA construct can be the same or can be different from the degree of complementarity between the antisense region of the siRNA and the target RNA sequence (e.g., DRAK2 RNA sequence). Complementarity to the target sequence of less than 100% in the antisense strand of the siRNA duplex (including deletions, insertions and point mutations) is reported to be tolerated when these differences are located between the 5′-end and the middle of the antisense siRNA (Elbashir et al., 2001, EMBO, 20(23):68-77-6888). Determination of binding free energies for nucleic acid molecules is well known in the art (e.g., see Turner et al., 1987, J. Am. Chem. Soc. 190:3783-3785; Frier et al., 1986 Proc. Nat. Acad. Sci. USA, 83: 9373-9377) “Perfectly complementary” means that all the contiguous residues of a nucleic acid molecule will hydrogen bond with the same number of contiguous residues in a second nucleic acid sequence. Appropriate hybridization conditions are well known to those skilled in the art, can be predicted readily based on sequence composition and conditions, or can be determined empirically by using routine testing (see Sambrook et al., (cf. Molecular Cloning: A Laboratory Manual, Third Edition, edited by Cold Spring Harbor Laboratory, 2000) at §§1.90-1.91, 7.37-7.57, 9.47-9.51 and 11.47-11.57, particularly at §§9.50-9.51, 11.12-11.13, 11.45-11.47 and 11.55-11.57). Sequences that are “sufficiently complementary” allow stable hybridization of a probe sequence to a target sequence, even if the two sequences are not completely identical.

A detection step may use any of a variety of known methods to detect the presence of nucleic acid by hybridization to a probe oligonucleotide. One specific example of a detection step uses a homogeneous detection method such as described in detail previously in Arnold et al. Clinical Chemistry 35:1588-1594 (1989), and U.S. Pat. Nos. 5,658,737 (Nelson et al.), and 5,118,801 and 5,312,728 (Lizardi et al.).

The types of detection methods in which probes can be used include Southern blots (DNA detection), dot or slot blots (DNA, RNA), and Northern blots (RNA detection). Labelled proteins could also be used to detect a particular nucleic acid sequence to which it binds (e.g., protein detection by far western technology: Guichet et al., 1997, Nature 385(6616): 548-552; and Schwartz et al., 2001, EMBO 20(3): 510-519). Other detection methods include kits containing reagents of the present invention on a dipstick setup and the like. Of course, it might be preferable to use a detection method which is amenable to automation. A non-limiting example thereof includes a chip or other support comprising one or more (e.g., an array) different probes.

A “label” refers to a molecular moiety or compound that can be detected or can lead to a detectable signal. A label is joined, directly or indirectly, to a nucleic acid probe or the nucleic acid to be detected (e.g., an amplified sequence). Direct labelling can occur through bonds or interactions that link the label to the nucleic acid (e.g., covalent bonds or non-covalent interactions), whereas indirect labelling can occur through the use of a “linker” or bridging moiety, such as additional oligonucleotide(s), which is/are either directly or indirectly labelled. Bridging moieties may amplify a detectable signal. Labels can include any detectable moiety (e.g., a radionuclide, ligand such as biotin or avidin, enzyme or enzyme substrate, reactive group, chromophore such as a dye or coloured particle, luminescent compound including a bioluminescent, phosphorescent or chemiluminescent compound, and fluorescent compound). In one particular embodiment, the label on a labelled probe is detectable in a homogeneous assay system, i.e., in a mixture, the bound label exhibits a detectable change compared to an unbound label.

Other methods of labelling nucleic acids are known whereby a label is attached to a nucleic acid strand as it is fragmented, which is useful for labelling nucleic acids to be detected by hybridization to an array of immobilized DNA probes (e.g., see PCT No. PCT/IB99/02073).

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural, rare or synthetic nucleotides. They can be designed to enhance a chosen criterion like stability, for example. Chimeras of deoxyribonucleotides and ribonucleotides may also be within the scope of the present invention.

“Amplification” refers to any known in vitro procedure for obtaining multiple copies (“amplicons”) of a target nucleic acid sequence or its complement or fragments thereof. In vitro amplification refers to the production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification, nucleic acid sequence-based amplification (NASBA), and strand-displacement amplification (SDA). Replicase-mediated amplification uses self-replicating RNA molecules, and a replicase such as Qβ-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Another known strand-displacement amplification method does not require endonuclease nicking (Dattagupta et al., U.S. Pat. No. 6,087,133). Transcription-mediated amplification (TMA) can also be used in the present invention. In one embodiment, TMA and NASBA isothermic methods of nucleic acid amplification are used. Those skilled in the art will understand that the oligonucleotide primer sequences of the present invention may be readily used in any in vitro amplification method based on primer extension by a polymerase (see generally Kwoh et al., 1990, Am. Biotechnol. Lab. 8:14 25 and (Kwoh et al., 1989, Proc. Natl. Acad. Sci. USA 86, 1173 1177; Lizardi et al., 1988, BioTechnology 6:1197 1202; Malek et al., 1994, Methods Mol. Biol., 28:253 260; and Sambrook et al., (cf. Molecular Cloning: A Laboratory Manual, Third Edition, edited by Cold Spring Harbor Laboratory, 2000). As commonly known in the art, the oligos are designed to bind to a complementary sequence under selected conditions.

As used herein, a “primer” defines an oligonucleotide which is capable of annealing to a target sequence, thereby creating a double stranded region which can serve as an initiation point for nucleic acid synthesis under suitable conditions. Primers can be, for example, designed to be specific for certain alleles so as to be used in an allele-specific amplification system. The primer's 5′ region may be non-complementary to the target nucleic acid sequence and include additional bases, such as a promoter sequence (which is referred to as a “promoter primer”). Those skilled in the art will appreciate that any oligomer that can function as a primer can be modified to include a 5′ promoter sequence, and thus function as a promoter primer. Similarly, any promoter primer can serve as a primer, independent of its functional promoter sequence. Of course the design of a primer from a known nucleic acid sequence is well known in the art. As for the oligos, it can comprise a number of types of different nucleotides.

As used herein, the twenty natural amino acids and their abbreviations follow conventional usage. Stereoisomers (e.g., D-amino acids) such as a,a-disubstituted amino acids, N-alkyl amino acids, lactic acid and other unconventional amino acids may also be suitable components for the polypeptides of the present invention. Examples of unconventional amino acids include but are not limited to selenocysteine, citrulline, ornithine, norvaline, 4-(E)-butenyl-4(R) methyl-N-methylthreonine (MeBmt), N-methyl-leucine (MeLeu), aminoisobutyric acid, statine, N-methyl-alanine (MeAla).

As used herein, “protein” or “polypeptide” means any peptide-linked chain of amino acids, regardless of post-translational modifications (e.g., acetylation, phosphorylation, glycosylation, sulfatation, sumoylation, prenylation, ubiquitination, etc). A “DRAK2 protein” or a “DRAK2 polypeptide” is an expression product of DRAK2 nucleic acid (e.g., DRAK2 gene) such as native human DRAK2 protein (SEQ ID NO:2), a DRAK2 protein homolog (e.g., mouse DRAK2, FIG. 13) that shares at least 60% (but preferably, at least 65, 70, 75, 80, 85, 86, 87, 88, 89, 90, 91, 92, 93, 94, 95, 96, 97, 98, 99, 100%) amino acid sequence identity with DRAK2 and displays functional activity of native DRAK2 protein. For the sake of brevity, the units (e.g., 66, 67 . . . 81, 82% . . . ) have not been specifically recited but are nevertheless considered within the scope of the present invention.

A “DRAK2 interacting protein” refers to a protein which binds directly or indirectly (e.g., via RNA or another bridging protein or molecule) to DRAK2 in order to modulate or participate in a functional activity of DRAK2. These proteins include kinases, phosphatases, scaffolding proteins, effector proteins, or any other proteins known to interact with DRAK2 (see below). An “isolated protein” or “isolated polypeptide” is purified from its natural in vivo state.

The terms “biological activity” or “functional activity” or “function” are used interchangeably and refer to any detectable biological activity associated with a structural, biochemical or physiological activity of a cell or protein (i.e. DRAK2). Other specific non-limiting examples of DRAK2 interacting proteins include kinases, phophatases and effector proteins. Therefore, interaction of DRAK2 with any of these DRAK2 interacting proteins is considered a functional activity of an DRAK2 protein. Thus, oligomerization of DRAK2 with specific proteins such as proteins containing SH2, domains as well as with itself is also considered a biological activity of DRAK2. Such interaction may be stable or transient. Another example of an DRAK2 functional activity is its capacity to become phosphorylated by several kinases. Thus, in accordance with the present invention, oligomerization and phosphorylation of DRAK2 are also considered as functional or biological activities of DRAK2. Interaction of DRAK2 with other known ligands (e.g., phophatases, effector proteins, etc) not explicitly listed in the present invention may also be considered functional activities of DRAK2. Thus, in accordance with the present invention, measuring the effect of a test compound on its ability to inhibit or increase (e.g., modulate) DRAK2 binding or interaction, level of expression as well as phosphorylation status is considered herein as measuring a biological activity of DRAK2.

As noted above, DRAK2 biological activity also includes any biochemical measurement of the protein, conformational changes, phosphorylation status (or any other posttranslational modification e.g., ubiquitination, sumolylation, palmytoylation, prenylation etc), any downstream effect of DRAK2's signalling such as protein phosphorylation in signalling cascades, indirect gene expression modulation, or any other feature of the protein that can be measured with techniques known in the art.

DRAK2. As used herein, the term “DRAK2 antibody” or “immunologically specific DRAK2 antibody” refers to an antibody that specifically binds to (interacts with) a DRAK2 protein and displays no substantial binding to other naturally occurring proteins other than the ones sharing the same antigenic determinants as the DRAK2 protein. DRAK2 antibodies include polyclonal, monoclonal, humanized as well as chimeric antibodies. Preferably these antibodies are cellular antibodies.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

As used herein, the designation “functional derivative” denotes, in the context of a functional derivative of an amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The substituting amino acid generally has chemico-physical properties, which are similar to that of the substituted amino acid. The similar chemico-physical properties include, similarities in charge, bulkiness, hydrophobicity, hydrophylicity and the like. The term “functional derivatives” is intended to include “segments”, “variants”, “analogs” or “chemical derivatives” of the subject matter of the present invention.

As used herein, “chemical derivatives” is meant to cover additional chemical moieties not normally part of the subject matter of the invention. Such moieties could affect the physico chemical characteristic of the derivative (i.e. solubility, absorption, half life and the like, decrease of toxicity). Such moieties are exemplified in Remington: The Science and Practice of Pharmacy by Alfonso R. Gennaro, 2003, 21st edition, Mack Publishing Company. Methods of coupling these chemical physical moieties to a polypeptide are well known in the art.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutation of nucleic acid molecule is a mutant nucleic acid molecule. A mutant polypeptide can be encoded from this mutant nucleic acid molecule.

The term “variant” refers herein to a protein, which is substantially similar in structure and biological activity to the protein, or nucleic acid of the present invention to maintain at least one of its biological activities. Thus, provided that two molecules possess a common activity and can substitute for each other, they are considered variants as that term is used herein, even if the composition, or secondary, tertiary or quaternary structure of one molecule is not identical to that found in the other, or if the amino acid sequence or nucleotide sequence is not identical. A homolog is a gene sequence encoding a polypeptide isolated from an organism other than a human being. Similarly, a homolog of a native polypeptide is an expression product of a gene homolog. Expression vectors, regulatory sequences (e.g., promoters), leader sequences and method to generate same and introduce them in cells are well known in the art.

Amino acid sequence variants of the polypeptides of the present invention (e.g., DRAK2) can be prepared by mutations in the DNA. Such variants include, for example, deletions from, or insertions or substitutions of, residues within the amino acid sequence shown in SEQ ID NOs: 2 or 4. Any combination of deletion, insertion, and substitution can also be made to arrive at the final construct, provided that the final construct possesses the desired activity.

While the site for introducing an amino acid sequence variation is predetermined, the mutation per se need not be predetermined. For example, to optimize the performance of a mutation at a given site, random mutagenesis can be conducted at the target codon or region and the expressed polypeptide (e.g., DRAK2) variants screened for the optimal combination of desired activity. Techniques for making substitution mutations at predetermined sites in DNA having a known sequence are well known in the art and include, for example, site-specific mutagenesis.

Preparation of a Variant in Accordance with the Present Invention is preferably achieved by site-specific mutagenesis of DNA that encodes an earlier prepared variant or a nonvariant version of the protein. Site-specific mutagenesis allows the production of variants through the use of specific oligonucleotide sequences that encode the DNA sequence of the desired mutation. In general, the technique of site-specific mutagenesis is well known in the art, as exemplified by publications such as Adelman et al., DNA 2:183 (1983) and Ausubel et al. “Current Protocols in Molecular Biology”, J. Wiley & Sons, NY, N.Y., 1996.

Amino acid sequence deletions generally range from about 1 to 30 residues, more preferably 1 to 10 residues, and typically are contiguous.

Amino acid sequence insertions include amino and/or carboxyl-terminal fusions of from one residue to polypeptides of essentially unrestricted length, as well as intrasequence insertions of single or multiple amino acid residues. Intrasequence insertions (i.e., insertions within the complete DRAK2) can range generally from about 1 to 10 residues, more preferably 1 to 5.

The third group of variants are those in which at least one amino acid residue in the DRAK2molecule, has been removed and a different residue inserted in its place. Such substitutions preferably are made in accordance with the following Table 1 when it is desired to modulate finely the characteristics of the polypeptide.

TABLE 1

| Original Residue | Exemplary Substitutions |
|---|---|
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | leu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Substantial changes in functional or immunological identity can be made by selecting substitutions that are less conservative than those in Table 1, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine.

Some deletions and insertions, and substitutions are not expected to produce radical changes in the characteristics of the polypeptides of the present invention. However, when it is difficult to predict the exact effect of the substitution, deletion, or insertion in advance of doing so, one skilled in the art will appreciate that the effect will be evaluated by routine screening assays. For example, a variant typically is made by site-specific mutagenesis of the native DRAK2 encoding-nucleic acid, expression of the variant nucleic acid in recombinant cell culture, and, optionally, purification from the cell culture, for example, by immunoaffinity adsorption on a column (to absorb the variant by binding it to at least one remaining immune epitope). The activity of the cell lysate or purified DRAK2 molecule variant is then screened in a suitable screening assay for the desired characteristic. For example, a change in the immunological character of the polypeptide molecule, such as affinity for a given antibody, is measured by a competitive type immunoassay. Changes in immunomodulation activity are measured by the appropriate assay. Modifications of such protein properties as redox or thermal stability, hydrophobicity, susceptibility to proteolytic degradation or the tendency to aggregate with carriers or into multimers are assayed by methods well known to the ordinarily skilled artisan.

Binding agent. A binding agent is a molecule or compound that specifically binds to or interacts with an DRAK2. Non-limiting examples of binding agents include antibodies, interacting partners, ligands, and the like. It will be understood that such binding agents can be natural, recombinant or synthetic.

In accordance with the present invention, it shall be understood that the “in vivo” experimental model (e.g., a transgenic animal of the present invention) can also be used to carry out an “in vitro” assay. For example, cellular extracts from the indicator cells can be prepared and used in one of the aforementioned “in vitro” tests (such as in binding assays or in vitro translation assays).

The term “subject” or “patient” as used herein refers to an animal, preferably a mammal, and most preferably a human who is the object of treatment, observation or experiment.

As used herein, the term “purified” refers to a molecule (e.g., DRAK2 polypeptide, antisense or RNAi molecule, etc) having been separated from a component of the composition in which it was originally present. Thus, for example, a “purified DRAK2 polypeptide or polynucleotide” has been purified to a level not found in nature. A “substantially pure” molecule is a molecule that is lacking in most other components (e.g., 30, 40, 50, 60, 70, 75, 80, 85, 90, 95, 96, 97, 98, 99, 100% free of contaminants). By opposition, the term “crude” means molecules that have not been separated from the components of the original composition in which it was present. Therefore, the terms “separating” or “purifying” refers to methods by which one or more components of the biological sample are removed from one or more other components of the sample. Sample components include nucleic acids in a generally aqueous solution that may include other components, such as proteins, carbohydrates, or lipids. A separating or purifying step preferably removes at least about 70% (e.g., 70, 75, 80, 85, 90, 95, 96, 97, 98, 99, 100%), more preferably at least about 90% (e.g., 90, 91, 92, 93, 94, 95, 96, 97, 98, 99, 100%) and, even more preferably, at least about 95% (e.g., 95, 96, 97, 98, 99, 100%) of the other components present in the sample from the desired component. For the sake of brevity, the units (e.g., 66, 67 . . . 81, 82, . . . 91, 92% . . . ) have not systematically been recited but are considered, nevertheless, within the scope of the present invention.

The terms “inhibiting,” “reducing” or any variation of these terms, when used in the claims and/or the specification includes any measurable decrease or complete inhibition of at least one biological activity of DRAK2 to achieve a desired result. For example, a compound is said to be inhibiting DRAK2 activity when a decrease of islet cells is measured following a treatment with the compounds of the present invention as compared to in the absence thereof. Other non-limiting examples include a reduction in the phosphorylation status of DRAK2.

As used herein, the terms “molecule”, “compound”, “agent” or “ligand” are used interchangeably and broadly to refer to natural, synthetic or semi-synthetic molecules or compounds. The term “compound” therefore denotes for example chemicals, macromolecules, cell or tissue extracts (from plants or animals) and the like. Non-limiting examples of compounds include peptides, antibodies, carbohydrates, nucleic acid molecules and pharmaceutical agents. The compound can be selected and screened by a variety of means including random screening, rational selection and by rational design using for example protein or ligand (e.g., S6 kinase which interact with DRAK2) modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, the modulating compounds of the present invention are modified to enhance their stability and their bioavailability. The compounds or molecules identified in accordance with the teachings of the present invention have a therapeutic value in diseases or conditions in which the physiology or homeostasis of the cell and/or tissue is compromised by DRAK2 production or response. For example, compounds of the present invention, by acting on a biological activity of DRAK2 (e.g., phosphorylation thereof) may decrease the function/activity thereof.

As used herein “antagonists”, “DRAK2 antagonists” or “DRAK2 inhibitors” refer to any molecule or compound capable of inhibiting (completely or partially) a biological activity of DRAK2. On the contrary, “agonists”, “DRAK2 agonists” or “DRAK2 stimulators” refer to any molecule or compound capable of enhancing or stimulating (completely or partially) a biological activity of DRAK2.

When referring to nucleic acid molecules, proteins or polypeptides, the term native refers to a naturally occurring nucleic acid or polypeptide. A homolog is a gene sequence encoding a polypeptide isolated from an organism other than a human being. Similarly, a homolog of a native polypeptide is an expression product of a gene homolog. Of course, the non-coding portion of a gene can also find a homolog portion in another organism.

As used herein, the term “pharmaceutically acceptable” refers to molecular entities and compositions that are physiologically tolerable and do not typically produce an allergic or similar untoward reaction, such as gastric upset, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans. The term “carrier” refers to a diluent, adjuvant, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E. W. Martin.

### Therapeutic Nucleic Acids

The present invention has identified DRAK2 as a target for the treatment of diabetes and autoimmune diseases. Thus, in one embodiment, the present invention generally relates to DRAK2 expression modulation and the use of DRAK2 expression modulation (i.e. DRAK2 expression inhibition) to treat or prevent onset or development of diabetes and autoimmune disease.

### SiRNAs

The present invention further concerns the use of RNA interference (RNAi) to decrease DRAK2 expression in target cells. “RNA interference” refers to the process of sequence specific suppression of gene expression mediated by small interfering RNA (siRNA) without generalized suppression of protein synthesis. While the invention is not limited to a particular mode of action, RNAi may involve degradation of messenger RNA (e.g., DRAK2 mRNA) by an RNA induced silencing complex (RISC), preventing translation of the transcribed targeted mRNA. Alternatively, it may involve methylation of genomic DNA, which shuts down transcription of a targeted gene. The suppression of gene expression caused by RNAi may be transient or it may be more stable, even permanent.

RNA interference is triggered by the presence of short interfering RNAs of about 20-25 nucleotides in length which comprise about 19 base pair duplexes. These siRNAs can be of synthetic origin or they can be derived from a ribonuclease III activity (e.g., dicer ribonuclease) found in cells. The RNAi response also features an endonuclease complex containing siRNA, commonly referred to as an RNA-induced silencing complex (RISC), which mediates the cleavage of single stranded RNA having a sequence complementary to the antisense region of the siRNA duplex. Cleavage of the target RNA (e.g., DRAK2 mRNA) takes place in the middle of the region complementary to the antisense strand of the siRNA duplex (Elbashir et al., 2001, Genes Dev., 15:188).

“Small interfering RNA” of the present invention refers to any nucleic acid molecule capable of mediating RNA interference “RNAi” or gene silencing (see for example, Bass, 2001, Nature, 411:428-429; Elbashir et al., 2001, Nature, 411:494-498; Kreutzer et al., International PCT publication No. WO 00/44895; Zernicka-Goetz et al., International PCT publication No. WO 01/36646; Fire, International PCT publication No. WO99/32619; Mello and Fire, International PCT publication No. WO01/29058; Deschamps-Depaillette, International PCT publication No. WO99/07409; Han et al., International PCT publication No. WO 2004/011647; Tuschl et al., International PCT publication No. WO 02/44321; and Li et al., International PCT publication No. WO 00/44914). For example, siRNA of the present invention are double stranded RNA molecules from about ten to about 30 nucleotides long that are named for their ability to specifically interfere with protein expression. In one embodiment, siRNA of the present invention are 12-28 nucleotides long, more preferably 15-25 nucleotides long, even more preferably 19-23 nucleotides long and most preferably 21-23 nucleotides long. Therefore preferred siRNA of the present invention are 12, 13, 14, 15, 16, 17, 18, 19, 20, 21, 22, 23, 24, 25, 26, 27, 28 nucleotides in length. As used herein, siRNA molecules need not to be limited to those molecules containing only RNA, but further encompass chemically modified nucleotides and non-nucleotides.

The length of one strand designates the length of a siRNA molecule. For example, a siRNA that is described as a 23 ribonucleotides long (a 23 mer) could comprise two opposite strands of RNA that anneal together for 21 contiguous base pairing. The two remaining ribonucleotides on each strand would form what is called an “overhang”. In a particular embodiment, the siRNA of the present invention contains two strands of different lengths. In this case, the longer strand designates the length of the siRNA. For example, a dsRNA containing one strand that is 20 nucleotides long and a second strand that is 19 nucleotides long is considered a 20 mer.

siRNAs that comprise an overhang are desirable. The overhang may be at the 3′ or 5′ end. Preferably, the overhangs are at the 3′ end of an RNA strand. The length of an overhang may vary but preferably is about 1 to 5 nucleotides long. Generally, 21 nucleotides siRNA with two nucleotides 3′-overhang are the most active siRNAs.

siRNA of the present invention are designed to decrease DRAK2 expression in a target cell by RNA interference. siRNA of the present invention comprise a sense region and an antisense region wherein the antisense region comprises a sequence complementary to an DRAK2 mRNA sequence (e.g., FIG. 16) and the sense region comprises a sequence complementary to the antisense sequence of DRAK2 mRNA. A siRNA molecule can be assembled from two nucleic acid fragments wherein one fragment comprises the sense region and the second fragment comprises the antisense region of siRNA molecule. The sense region and antisense region can also be covalently connected via a linker molecule. The linker molecule can be a polynucleotide linker or a non-polynucleotide linker.

In one embodiment, the present invention features a siRNA molecule having RNAi activity against DRAK2 RNA, wherein the siRNA molecule comprises a sequence complementary to any RNA having an DRAK2 encoding sequence. A siRNA molecule of the present invention can comprise any contiguous DRAK2 sequence (e.g., 19-23 contiguous nucleotides present in a DRAK2 sequence such as shown in SeQ ID NO:1).

siRNAs of the present invention comprise a ribonucleotide sequence that is at least 80% identical to an DRAK2 ribonucleotide sequence. Preferably, the siRNA molecule is at least 90%, at least 95% (e.g., 95, 96, 97, 99, 99, 100%), at least 98% (e.g., 98, 99, 100%) or at least 99% (e.g., 99, 100%) identical to the ribonucleotide sequence of the target gene (e.g., DRAK2 RNA). siRNA molecule with insertion, deletions, or single point mutations relative to the target may also be effective. Mutations that are not in the center of the siRNA molecule are more tolerated. Tools to assist siRNA design are well known in the art and readily available to the public. For example, a computer-based siRNA design tool is available on the Internet at www.dharmacon.com or on the web site of several companies that offer the synthesis of siRNA molecules.

In one embodiment, the siRNA molecules of the present invention are chemically modified to confer increased stability against nuclease degradation but retain the ability to bind to the target nucleic acid that is present in a cell. Modified siRNAs of the present invention comprise modified ribonucleotides, and are resistant to enzymatic degradation such as RNAse degradation, yet they retain their ability to reduce DRAK2 expression in a target cell. The siRNA may be modified at any position of the molecule so long as the modified siRNA is still capable of binding to the target sequence and is more resistant to enzymatic degradation. Modifications in the siRNA may be in the nucleotide base (i.e., purine or pyrimidine), the ribose or phosphate.

More specifically, the siRNA may be modified in at least one purine, in at least one pyrimidine or a combination thereof. Generally, all purines (adenosine or guanine) or all pyrimidine (cytosine or uracyl) or a combination of all purines and all pyrimidines of the siRNA are modified. Ribonucleotides on either one or both strands of the siRNA may be modified.

Non-limiting examples of chemical modification that can be included in an siRNA molecule include phosphorothioate internucleotide linkages (see US 2003/0175950), 2′-O-methyl ribonucleotides, 2′-O-methyl modified ribonucleotides, 2′-deoxy-2′-fluoro ribonucleotides, 2′-deoxy-2′-fluoro modified pyrimidines nucleotides, 5-C-methyl nucleotides and deoxyabasic residue incorporation. The ribonucleotides containing pyrimidine bases can be modified at the 2′ position of the ribose residue. A preferable modification is the addition of a molecule from the halide chemical group such as fluorine. Other chemical moieties such as methyl, methoxymethyl and propyl may also be added as modifications (see International PCT publication No. WO2004/011647). These chemical modifications, when used in various siRNA constructs, are shown to preserve RNAi activity in cells while at the same time, dramatically increasing their stability in cells or serum. Chemical modifications of the siRNA of the present invention can also be used to improve the stability of the interaction with the target RNA sequence.

siRNAs of the present invention may also be modified by the attachment of at least one receptor binding ligand to the siRNA. Receptor binding ligand can be any ligand or molecule that directs the siRNA of the present invention to a specific target cell (e.g., NK cells, macrophage, dendritic cells). Such ligands are useful to direct delivery of siRNA to a target cell in a body system, organ or tissue of a subject such as NK cells. Receptor binding ligand may be attached to one or more siRNA ends, including any combination of 5′ or 3′ ends. The selection of an appropriate ligand for delivering siRNAs depends on the cells, tissues or organs that are targeted and is considered to be within the ordinary skill of the art. For example, to target a siRNA to hepatocytes, cholesterol may be attached at one or more ends, including 3′ and 5′ ends. Other conjugates such as other ligands for cellular receptors (e.g., peptides derived from naturally occurring protein ligands), protein localization sequences (e.g., ZIP code sequences), antibodies, nucleic acid aptamers, vitamins and other cofactors such as N-acetylgalactosamine and folate, polymers such as polyethyleneglycol (PEG), polyamines (e.g., spermine or spermidine) and phospholipids can be linked (directly or indirectly) to the siRNA molecule for improving its bioavailability.

siRNAs can be prepared in a number of ways well known in the art, such as by chemical synthesis, T7 polymerase transcription, or by treating long double stranded RNA (dsRNA) prepared by one of the two previous methods with Dicer enzyme. Dicer enzyme create mixed population of dsRNA from about 21 to 23 base pairs in length from double stranded RNA that is about 500 base pairs to about 1000 base pairs in size. Dicer can effectively cleave modified strands of dsRNA, such as 2′-fluoromodified dsRNA (see WO2004/011647).

In one embodiment, vectors are employed for producing siRNAs by recombinant techniques. Thus, for example, a DNA segment encoding a siRNA derived from an DRAK2 sequence (e.g., FIG. 16) may be included in any one of a variety of expression vectors for expressing any DNA sequence derived from an DRAK2 sequence. Such vectors include synthetic DNA sequences (e.g., derivatives of SV40, bacterial plasmids, baculovirus, yeast plasmids, viral DNA such as vaccinia, fowl pox virus, adenovirus, lentivirus, retrovirus, adeno-associated virus, alphavirus etc.), chromosomal and non-chromosomal vectors. Any vector may be used in accordance with the present invention as long as it is replicable and viable in the desired host. The DNA segment in the expression vector is operatebly linked to an appropriate expression control sequence (e.g., promoter) to direct siRNA synthesis. Preferably, the promoters of the present invention are from the type III class of RNA polymerase III promoters (e.g., U6 and H1 promoters). The promoters of the present invention may also be inducible, in that the expression may be turned on or turned off (e.g., tetracycline-regulatable system employing the U6 promoter to control the production of siRNA targeted to DRAK2).

In a particular embodiment, the present invention utilizes a vector wherein a DNA segment encoding the sense strand of the RNA polynucleotide is operatebly linked to a first promoter and the antisense strand of the RNA polynucleotide is operably linked to a second promoter (i.e., each strand of the RNA polynucleotide is independently expressed).

In another embodiment, the DNA segment encoding both strands of the RNA polynucleotide is under the control of a single promoter. In a particular embodiment, the DNA segment encoding each strand is arranged on the vector with a loop region connecting the two DNA segments (e.g., sense and antisense sequences), where the transcription of the DNA segments and loop region creates one RNA transcript. When transcribed, the siRNA folds back on itself to form a short hairpin capable of inducing RNAi. The loop of the hairpin structure is preferably from about 4 to 6 nucleotides in length. The short hairpin is processed in cells by endoribonucleases which remove the loop thus forming a siRNA molecule. In this particular embodiment, siRNAs of the present invention comprising a hairpin or circular structure are about 35 to about 65 nucleotides in length (e.g., 35, 36, 37, 38, 49, 40, 41, 42, 43, 44, 45, 46, 47, 48, 49, 50, 51, 52, 53, 54, 55, 56, 57, 58, 59, 60, 61, 63, 64, 65 nucleotides in length), preferably between 40 and 64 nucleotides in length comprising for example about 18, 19, 20, 21, 22, or 23, 24, 25 base pairs.

In yet a further embodiment, the vector of the present invention comprises opposing promoters. For example, the vector may comprise two RNA polymerase III promoters on either side of the DNA segment (e.g., a specific DRAK2 DNA segment) encoding the sense strand of the RNA polynucleotide and placed in opposing orientations, with or without a transcription terminator placed between the two opposing promoters.

Non-limiting examples of expression vectors used for siRNA expression are described in Lee et al., 2002, Nature Biotechnol., 19:505; Miyagishi and Taira, 2002, Nature Biotechnol., 19:497; Pau et al., 2002, Nature Biotechnol., 19:500 and Novina et al., 2002, Nature Medecine, July 8(7):681-686).

Numerous methods of designing siRNAs are known to the skill artisan. Non-limiting examples include the Ambion system of Applied Biosystems, Technical Bulletin #506, the system of Invitrogen or as described in Reynolds et al., 2004.

### Antisense RNAs

The present invention also features antisense nucleic acid molecules which can be used for example to decrease or abrogate the expression of DRAK2 to increase the protection of islet cells. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845, and U.S. Pat. No. 5,593,974. Antisense nucleic acid molecules according to the present invention can be derived from the nucleic acid sequences and modified in accordance with well-known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

In one embodiment, antisense approach of the present invention involves the design of oligonucleotides (either DNA or RNA) that are complementary to DRAK2 mRNA. The antisense oligonucleotides bind to DRAK2 mRNA and prevent its translation. Absolute complementarity, although preferred, is not a definite prerequisite. One skilled in the art can identify a certain tolerable degree of mismatch by use of standard methods to determine the melting point of the hybridized antisense complex. In general, oligonucleotides that are complementary to the 5′ untranslated region (up to the first AUG initiator codon) of DRAK2 mRNA should work more efficiently at inhibiting translation and production of DRAK2 protein. However, oligonucleotides that are targeted to a coding portion of the sequence may produce inactive truncated protein or diminish the efficiency of translation thereby lowering the overall expression of DRAK2 protein in a cell. Antisense oligonucleotides targeted to the 3′ untranslated region of messages have also proven to be efficient in inhibiting translation of targeted mRNAs (Wagner, R. (1994), Nature, 372:333-335). The DRAK2 antisense oligonucleotides of the present invention are less than 100 nucleotides in length, particularly, less than 50 nucleotides in length and more particularly less than 30 nucleotides in length. Generally, effective antisense oligonucleotides are at least 15 or more oligonucleotides in length.

The antisense oligonucleotides of the present invention can be DNA, RNA, Chimeric DNA-RNA analogue, and derivatives thereof (see Inoue et al. (1987), Nucl. Acids. Res. 15: 6131-6148; Inoue et al. (1987), FEBS lett. 215: 327-330; Gauthier at al. (1987), Nucl. Acids, Res. 15: 6625-6641.). As mentioned above, antisense oligonucleotides of the present invention may include modified bases or sugar moiety. Examples of modified bases include xanthine, hypoxanthine, 2-methyladenine, N6-isopentenyladenine, 2-methylguanine, 3-methylcytosine, 5-methylcytosine, N6-adenine, 7-methyguanine, 5-fluorouracil, 5-chlorouracil, 5-bromouracil, 5-iodouracyl, 5-carboxymethylaminomethyluracil, 5-methoxycarboxymethyluracil, queosine, 4-thiouracil and 2,6-diaminopurine. Examples of modified sugar moieties include hexose, xylulose, arabinose and 2-fluoroarabinose. The antisense oligonucleotides of the present invention may also include modified phosphate backbone such as methylphosphonate, phosphoramidate, phosphoramidothioates, phosphordiamidate and alkyl phosphotriesters. The synthesis of modified oligonucleotides can be done according to methods well known in the art.

Once an antisense oligonucleotide or siRNA is designed, its effectiveness can be appreciated by conducting in vitro studies that assess the ability of the antisense to inhibit gene expression (e.g., DRAK2 protein expression). Such studies ultimately compare the level of DRAK2 RNA or protein with the level of a control experiment (e.g., an oligonucleotide which is the same as that of antisense experiment but being a sense oligonucleotide or an oligonucleotide of the same size as the antisense oligonucleotide but that does not bind to a specific DRAK2 sequence).

### Gene Therapy Methods

In the gene therapy methods of the present invention, an exogenous sequence (e.g., an DRAK2 gene or cDNA sequence, an DRAK2 siRNA or antisense nucleic acid) is introduced and expressed in an animal (preferably a human) to supplement, replace or inhibit a target gene (i.e., DRAK2), or to enable target cells to produce a protein (e.g., a DRAK2 dominant negative mutant) having a prophylactic or therapeutic effect toward diabetes and other DRAK2 related diseases.

Non virus-based and virus-based vectors (e.g., adenovirus- and lentivirus-based vectors) for insertion of exogenous nucleic acid sequences into eukaryotic cells are well known in the art and may be used in accordance with the present invention. Virus-based vectors (and their different variations) for use in gene therapy are well known in the art. In virus-based vectors, parts of a viral gene are replaced by the desired exogenous sequence so that a viral vector is produced. Viral vectors are very often designed to no longer be able to replicate due to DNA manipulations.

In one specific embodiment, lentivirus derived vectors are used to target an DRAK2 sequence (e.g., siRNA, antisense, nucleic acid encoding a partial or complete DRAK2 protein) into specific target cells (e.g., islet cells). These vectors have the advantage of infecting quiescent cells (for example see U.S. Pat. No. 6,656,706; Amado et al., 1999, Science 285: 674-676).

In addition to an DRAK2 nucleic acid sequence, siRNA or antisense, the vectors of the present invention may contain a gene that acts as a marker by encoding a detectable product.

One way of performing gene therapy is to extract cells from a patient, infect the extracted cells with a viral vector and reintroduce the cells back into the patient. A selectable marker may or may not be included to provide a means for enriching the infected or transduced cells. Alternatively, vectors for gene therapy that are specially formulated to reach and enter target cells may be directly administered to a patient (e.g., intravenously, orally etc.).

The exogenous sequences (e.g., antisense RNA, siRNA, a DRAK2 sequence, or DRAK2 targeting vector for homologous recombination) may be delivered into cells that express DRAK2 according to well known methods. Apart from infection with virus-based vectors, examples of methods to deliver nucleic acid into cells include DEAE dextran lipid formulations, liposome-mediated transfection, CaCl₂-mediated transfection, electroporation or using a gene gun. Synthetic cationic amphiphilic substances, such as dioleoyloxypropylmethylammonium bromide (DOTMA) in a mixture with dioleoylphosphatidylethanolamine (DOPE), or lipopolyamine (Behr, Bioconjugate Chem., 1994 5:382), have gained considerable importance in charged gene transfer. Due to an excess of cationic charge, the substance mixture complexes with negatively charged genes and binds to the anionic cell surface. Other methods include linking the exogenous oligonucleotide sequence (e.g., siRNA, antisense, DRAK2 sequence encoding an DRAK2 protein, DRAK2 targeting vector for homologous recombination, etc.) to peptides or antibodies that especially bind to receptors or antigens at the surface of a target cell. U.S. Pat. No. 6,358,524 describes target cell-specific non-viral vectors for inserting at least one gene into cells of an organism. The method describes the use of non-viral carriers that are cationized to enable them to complex with the negatively charged DNA.

To achieve high cellular concentration of the DRAK2 antisense nucleic acid or small inhibitor RNAs of the present invention, an effective method utilizes a recombinant DNA construct in which the nucleic acid sequence is placed under a strong promoter and the entire construct is targeted into the cell. Such promoter may constitutively or inducibly produce the DRAK2 sequence encoding DRAK2 protein (or portion thereof), antisense RNA or siRNA of the present invention.

### Assays to Identify Modulators of DRAK2

In order to identify modulators (preferably inhibitors) of DRAK2, several screening assays aiming at reducing, abrogating or stimulating a functional activity of DRAK2 in cells can be designed in accordance with the present invention.

One possible way is by screening libraries of candidate compounds for inhibitors of the phosphorylation of DRAK2.

For example, combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection may be used in order to identify modulators of DRAK2 biological activity. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261:1303; Carrell et al. (1994) Angew. Chem., Int. Ed Engl. 33:2059; and ibid 2061; and in Gallop et al. (1994). Med. Chem. 37:1233. Libraries of compounds may be presented in solution (e.g., Houghten (1992) Biotechniques 13:412-421) or on beads (Lam (1991) Nature 354:82-84), chips (Fodor (1993) Nature 364:555-556), bacteria or spores (Ladner U.S. Pat. No. 5,223,409), plasmids (Cull et al. (1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) supra; Erb et al. (1994) supra; Zuckermann et al., (1994) supra; Cho et al. (1993) supra; Carrell et al. (1994) supra, or luciferase, and the enzymatic label detected by determination of conversion of an appropriate substrate to product. The choice of a particular combinatorial library depends on the specific DRAK2 activity that needs to be modulated.

All methods and assays of the present invention may be developed for low-throughput, high-throughput, or ultra-high throughput screening formats. Of course, methods and assays of the present invention are amenable to automation. Automation and low-throughput, high-throughput, or ultra-high throughput screening formats are possible for the screening of agents which modulates the level and/or activity of DRAK2.

Generally, high throughput screens for DRAK2 modulators i.e. candidate or test compounds or agents (e.g., peptides, peptidomimetics, small molecules, antisense RNA, Ribozyme, or other drugs) may be based on assays which measure a biological activity of DRAK2. The invention therefore provides a method (also referred to herein as a “screening assay”) for identifying modulators, which have an inhibitory effect on, for example, an DRAK2 biological activity or expression thereof, or which binds to or interacts with DRAK2 proteins, or which has an inhibitory effect on islet cells apoptosis.

The assays described above may be used as initial or primary screens to detect promising lead compounds for further development. Often, lead compounds will be further assessed in additional, different screens. Therefore, this invention also includes secondary DRAK2 screens which may involve assays utilizing mammalian cell lines expressing DRAK2.

Tertiary screens may involve the study of the identified modulators in the appropriate rat and mouse models. Accordingly, it is within the scope of this invention to further use an agent identified as described herein in an appropriate animal model. For example, a test compound identified as described herein (e.g., an DRAK2 inhibiting agent, an antisense DRAK2 nucleic acid molecule, an DRAK2 siRNA, an DRAK2 antibody etc.) can be tested in the transgenic mice overexpressing DRAK2 of the present invention to determine the efficacy, toxicity, or side effects of treatment with such an agent. Furthermore, this invention pertains to uses of novel agents identified by the above-described screening assays for treatment of cancers, infectious diseases and autoimmune diseases, as described herein.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1—Drak2 was rapidly augmented in islets treated with FFA. A. Drak2 mRNA expression according to real time RT-PCR. Islets were stimulated by FFA (0.7 mM oleate and palmitate mixed in a 2:1 ratio) in vitro, or C57BL/6 mice were injected with 15 mM FFA 0.5 ml (oleate and palmitate mixed in a 2:1 ratio) i.v. in PBS for the indicated durations. For the in vivo experiment, the time indicated was from the time of FFA injection until sacrifice of mice. The duration of islet isolation (about 1 h) was not calculated in. Drak2 mRNA expression in islet cells was measured by real time RT-PCR. The ratio of Drak2 mRNA and β-actin mRNA was taken as a measure of Drak2 mRNA levels. The samples were in triplicate, and the means+SD of 4-6 independent experiments are shown. B and C-Drak2 protein expression according to flow cytometry. C57BL/6 islets were cultured for 48 h in the absence or presence of FFA as described above. The islets were dispersed after the culture and analyzed by 2-color flow cytometry for intracellular insulin and Drak2. The experiment was repeated 4 times. A representative set of histograms is shown in FIG. 1B and the summary of all 4 experiments is illustrated in FIG. 1C. The asterisk indicates a p value of <0.01 according to Student's t test.

FIG. 2—Drak2 siRNA inhibited Drak2 protein upregulation and reduced apoptosis in NIT-1 cells upon FFA stimulation. A. Drak2 protein levels in NIT-1 cells. NIT-1 cells were transfected with 2 Drak2 siRNAs (#592 and #1162), or a control siRNA, (a scrambled sequence of #1162). The cells were cultured for 24 h in the absence or presence of FFA, as indicated, and then analyzed for intracellular Drak2 protein levels by flow cytometry. The experiment was repeated 4-5 times, and means+SD of these experiments are shown. B. Drak2 siRNA prevented FFA-induced apoptosis in NIT-1 cells. NIT-1 cells were transfected with the same 2 Drak2 siRNAs (#592 and #1162), or a control siRNA. The cells were cultured for 24 h in the absence or presence of FFA, as indicated, and analyzed for apoptosis by flow cytometry with annexin V staining. The experiment was repeated 4-5 times, and means+SD of percentage apoptosis of all of these experiments are shown.

FIG. 3—Drak2 overexpression in Tg islet β-cells. Drak2 Tg or WT islets were analyzed by 2-color flow cytometry for Drak2 and insulin expression (right column). The percentage of Drak2 positive cells among insulin-positive cells and their mean fluorescent intensity (MFI) are indicated in the left column. Upper row, WT; bottom row, Drak2 Tg.

FIG. 4—Drak2 Tg islets were prone to apoptosis upon FFA stimulation. A and B. Flow cytometry analysis of islet cell apoptosis. Drak2 Tg and WT islets were cultured in RPMI1640 with 10% FCS and stimulated with FFA, as described in FIG. 1. After 16 h or 48 h, as indicated, the islets were dispersed and analyzed by flow cytometry with annexin V staining. The percentage of annexin V-positive cells is shown in the histograms (FIG. 4A). The experiment was repeated 3-6 times and the mean+SD of all these experiments are illustrated in FIG. 4B. The asterisk indicates p<0.05, according to Student's t test. C. Islet insulin release after FFA stimulation. Islets from Tg or WT mice were cultured in F-12K medium with 10% FCS in the presence or absence of FFA, as described in FIG. 1. Insulin release measurements by these cells were conducted after 48 h. For each treatment, the fold increase between low glucose and high glucose stimuli was first calculated. The fold increase of insulin release by the controls (i.e., WT or Tg islets cultured in the absence of FFA) was considered as 100% for its respective group (i.e., Tg or WT). Fold increase of FFA-treated islets upon high glucose stimulation was expressed as a percentage of the controls. After arcsine angular transformation of the percentage, Student's t test was conducted. Insulin release by Tg islets after FFA stimulation was significantly lower than that by WT islets (p<0.05).

FIG. 5—Compromised anti-apoptotic factor upregulation in Drak2 Tg islets. Drak2 and WT islets were stimulated by FFA as described in FIG. 1. The islets were harvested after 24 h, and their Bcl-2, Bcl-xL and Flip mRNA was measured by real-time RT-PCR. The samples were in triplicate. Means+SD of the ratios of signals of these molecules versus those of β-actin from 2 independent experiments are shown.

FIG. 6—Drak2 Tg islets were prone to apoptosis upon inflammatory and FFA stimulation. A-D. Flow cytometry analysis of islet cell apoptosis. Drak2 Tg and WT islets were cultured in RPMI1640 with 10% FCS and stimulated with STZ, IFN-γ plus IL-1β, TNF-α plus IL-1β, or FFA as described in FIG. 1. After 16 h or 48 h, as indicated, the islets were dispersed and analyzed by flow cytometry with annexin V staining. The percentage of annexin V-positive cells is shown in the histograms. The experiment was repeated more than twice, and a representative set of data is shown. E. Insulin release assay of islets after cytokine and FFA stimulation. Islets from Tg or WT mice were cultured in F-12K medium with 10% FCS in the presence or absence of IFN-γ plus IL-1β, TNF-α plus IL-1β, or FFA as described in FIG. 1. Insulin release by these cells as conducted after 48 h. Means±SD of results from 2 independent experiments are shown. For each treatment, the fold increase between low glucose and high glucose was first calculated. The fold increase of insulin release by islets in medium was used as a reference (considered as 100%) for its respective group (i.e., Tg or WT), and fold increase of each treatment was expressed as a percentage of the reference to its group. After arcsine angular transformation of the percentage, Student's t test was conducted. Insulin release by Tg islets after IFN-γ plus IL-1β, TNF-α plus IL-1β, or FFA stimulation was all significantly lower than that by WT islets (p<0.05, p<0.05 and p<0.01, respectively).

FIG. 7—Compromised anti-apoptotic factor upregulation in Drak2 Tg islets. Drak2 and WT islets were stimulated by IFN-γ plus IL-1β, TNF-α plus IL-1β, or FFA as described in FIG. 1. The islets were harvested after 24 h, and their Bcl-2, Bcl-xL and Flip mRNA was measured by real-time RT-PCR. The samples were in triplicate. Means±SD of the ratios of signals of these molecules versus those of β-actin from 2 independent experiments are shown.

FIG. 8—Increased diabetes risk in Drak2 Tg mice. A. Increased diabetes incidence in Drak2 Tg mice treated with multiple-low-dose STZ. Tg and WT mice were injected with multiple-low-dose STZ (40 mg/kg body weight, i.p., q.d. for 5 days). Blood glucose was monitored on the days indicated. Means±SEM are shown. On days 12 and 15 (marked with arrows), diabetes incidence in Tg mice was significantly higher than in WT mice (n=8 pairs; paired Student's t test, p<0.05). B. Reduced glucose tolerance in Drak2 Tg mice after diet-induced obesity. Drak2 Tg and WT mice were fed a high-fat diet for 6 weeks from 9 weeks of age. Both groups became obese at age 15 weeks when the glucose tolerance test was conducted. Tg mice on a high-fat diet presented significantly higher blood glucose at 30, 60, and 90 min after i.p. glucose injection, compared to WT mice (n=6 pairs, p<0.05, paired Student's t test).

FIG. 9—Drak2 mRNA was rapidly augmented in islets encountering inflammatory stimulation. Drak2 mRNA expression in C57BL/6 islet cells was measured by real time RT-PCR. The ratio of Drak2 mRNA and β-actin mRNA was taken as a measure of Drak2 mRNA levels. The samples were in triplicate, and the means+SD of 5 to 6 independent experiments are shown. A. Islets were stimulated by IFN-γ (1,000 U/ml) plus IL-1β (0.5 ng/ml) in vitro for 24 h. B. Islets were stimulated by TNF-α (200 ng/ml) plus IL-1β (0.5 ng/ml) in vitro for 24 h.

FIG. 10—Drak2 protein upregulation in β-cells upon inflammatory stimuli and its correlation to β cell apoptosis. A. Flow cytometry analysis of Drak2 protein expression in islet β-cells. C57BL/6 islets were cultured for 48 h in the absence or presence of IFN-γ plus IL-1β, or TNF-α plus IL-1β as described in FIG. 1. The islets were dispersed after culture and analyzed by 2-color flow cytometry for intracellular insulin and Drak2. The experiment was repeated 4 times. The means+SD of 4 experiments are illustrated. Asterisks indicate p values (<0.01 or <0.05) according to Student's t test. B. Drak2 siRNA inhibited Drak2 protein upregulation in NIT-1 insulinoma cells. NIT-1 insulinoma cells were transfected with Drak2 siRNA or control siRNA. The cells were cultured for 24 h in the absence or presence of IFN-γ plus IL-1β, or TNF-α plus IL-1β as described in FIG. 1, and then analyzed for intracellular Drak2 protein levels by flow cytometry. The experiment was repeated 4 to 5 times (n=4 or n=5, as indicated), and means+SD of these experiments are shown. Asterisks indicate p values (<0.05) of siRNA-versus control siRNA-treated cells, according to Student's t test. C. Inhibition of Drak2 expression by siRNA prevented cytokine-induced apoptosis in NIT-1 cells. NIT-1 cells were transfected with Drak2 siRNA or control siRNA. The cells were cultured for 24 h in the absence or presence of IFN-γ plus IL-1β, or TNF-α plus IL-1β as described in FIG. 1, and analyzed for apoptosis by flow cytometry with annexin V staining. The experiment was repeated 4 to 5 times (n=4 or n=5, as indicated), and means+SD of percentage of apoptosis in all these independent experiments are shown. Asterisks indicate p values (<0.05) of siRNA-versus control siRNA-treated cells, according to Student's t test.

FIG. 11—Drak2 overexpression in Tg islet β-cells. A. Drak2 mRNA overexpression in Tg islets. Islets from actin promoter-driven Drak2 Tg mice or their WT littermates were isolated and Drak2 mRNA levels were measured by real time RT-PCR. The samples were in triplicate. Means+SD of Drak2/β-actin mRNA ratios of 2 independent experiments are shown. B. Drak2 protein overexpression in Tg β-cells. Drak2 Tg or WT islets were analyzed by confocal microscopy for Drak2 and insulin expression. The Drak2 signal is in green, and insulin, in red. Representative data from 2 experiments are shown.

FIG. 12—Drak2 Tg islets were prone to apoptosis upon inflammatory cytokine stimulation. A-C. Flow cytometry analysis of islet cell apoptosis. Drak2 Tg and WT islets were cultured in RPMI 1640 medium with 10% FCS and stimulated with IFN-γ (1000 U/ml) plus IL-1β (0.5 ng/ml) or TNF-α (200 ng/ml) plus IL-1β (0.5 ng/ml). After 48 h, the islets were dispersed and analyzed by flow cytometry with annexin V staining. The percentage of annexin V-positive cells is shown in the histograms. The experiment was repeated more than 4-6 times. A representative set of data is shown in FIGS. 12A and 12B, and a summary of all the experiments appears in FIG. 12C, with the number of experiments (n) indicated. Asterisks indicate p<0.05 according to paired Student's t test. D. Insulin release assay of islets after cytokine stimulation. Islets from Tg or WT mice were cultured in the presence or absence of IFN-γ (1000 U/ml) plus IL-1β (0.5 ng/ml) or TNF-α (200 ng/ml) plus IL-1β (0.5 ng/ml). Insulin release by these islets (10 islets/treatment/well) was measured after 48 h. Samples were in duplicate. Means+SD of the results of 4 determinants from 2 independent experiments are shown in terms of fold increase in insulin release stimulated by 16.7 mM versus 2.8 mM glucose. E. Increased type 1 diabetes incidence in mice with transplanted Drak2 Tg islets. Diabetes was induced in C57BL/6 mice by a single i.p. STZ injection (200 mg/kg body weight). After 14 days, the diabetes status of these mice was confirmed according to blood glucose levels. WT or Tg islets were then transplanted i.p. to these diabetic mice to achieve euglycemia. After another 14 days, the glucose tolerance of these mice was verified to be similar (data now shown). Multiple low doses of STZ (40 mg/kg body weight/day×5 days) were subsequently given i.v. to these islet transplant recipients. Their blood glucose levels from day 0 (the day after multiple low doses of STZ injection was terminated) to day 18 are shown. From days 15 on, the blood glucose levels of the Tg and WT islet recipients are significantly different (p<0.05, Student's t test).

FIG. 13—p70S6 kinase phosphorylation by Drak 2 in vitro. A and B. Generation of recombinant GST-Drak2. GST-Drak2 was produced in E. coli with the construct pGEX-4T-1-Drak2 (FIG. 13A). The recombinant protein was first affinity-purified with glutathione-agarose beads, followed by size-exclusion chromatography. The purified protein appeared at the expected size (71 kD) and was more that 95% pure according to Coomassie Blue (left lane, FIG. 13B) and silver staining (middle lane, FIG. 13B). In some experiments, the GST tag of GST-Drak2 was cleaved by thrombin during affinity purification, and the purity of the untagged Drak2 was more than 95%, according to Coomassie Blue staining (right lane, FIG. 13B). C. GST-Drak2 was kinase-active. GST-Drak2 was employed in an in vitro kinase assay. The product of the assay was resolved by 12% SDS-PAGE, followed by autoradiography. A distinct radio-labeled band at the expected size of GST-Drak2 (71 kD) was detected. D and E. Generation of recombinant GST-p70S6 kinase. GST-p70S6 kinase was produced in E. coli with the construct pGEX-4T-1-p70S6K (FIG. 13D). The recombinant protein was first affinity purified with glutathione-agarose beads, followed cleavage of the GST-tag by thrombin. The purified protein appeared at the expected size with more than 95% purity, according to Coomassie Blue staining (FIG. 13E). F. p70S6 kinase phosphorylated by Drak2 in vitro. Mouse recombinant Drak2 and p70S6 kinase were reacted in an in vitro kinase assay. The product of the reaction was resolved by 12% SDS-PAGE, followed by autoradiography. Distinct radio-labeled bands at the expected sizes of Drak2 and p70S6 kinase were detected (lane 1). In lane 2, p70S6 kinase alone was present in the in vitro kinase assay without Drak2, and no radioactive band was detected.

FIG. 14—Drak 2 phosphorylation p70S6 kinase in vivo. A and B. Expression of HA-Drak2 in NIT-1 cells. NIT-1 cells were transiently transfected with pCEP-HA-Drak2 (FIG. 14A). After 48 h, recombinant HA-Drak2 was affinity-purified from the cell lysates with anti-HA agarose, followed by HA peptide elution. The purified protein was resolved in 12% SDS-PAGE, and immunoblotted with anti-HA Ab (FIG. 14B). Left lane: protein purified from pCEP-HA-Drak2-transfected NIT-1 cells; right lane: protein purified from empty vector pCEP-HA transfected NIT-1 cells using the same procedure. C. Recombinant HA-Drak2 was kinase-active. HA-Drak2, affinity-purified from in pCEP-HA-Drak2-transfected NIT-1 cells, was employed in an in vitro kinase assay. The product of the assay was resolved by 12% SDS-PAGE, followed by autoradiography. A distinct radio-labeled band at the expected size of HA-Drak2 was detected (left lane). No radio-labeled band was detected using a sample purified from empty vector-transfected NIT-1 cells (right lane). D. Drak2 overexpression led to enhanced p70S6 kinase phosphorylation in vivo. NIT-1 cells were transiently transfected with pCEP4-HA-Drak2 (left lane) or empty vector pCEP4-HA (right lane). After 48 h, the cells were harvested, and the lysates were analyzed with immunoblotting. Upper panel: the membrane was blotted with anti-HA to ascertain the HA-Drak2 overexpression; middle panel: the membrane was blotted with anti-phospho-p70S6 kinase to assess p70S6 kinase phosphorylation; bottom panel: the membrane was blotted with anti-p70S6 kinase to ascertain the similar total p70S6 kinase levels in NIT-1 cells transfected with pCEP4-HA-Drak2 or the empty vector pCEP4-HA.

FIG. 15—Effect of Drak2 siRNA on p70S6 kinase phosphorylation and effect of rapamycin on β-cell apoptosis. A-C. Drak2 siRNA inhibited p70S6 kinase phosphorylation in vivo. NIT-1 cells were stimulated with IFN-γ. (1000 U/ml) plus IL-1β (0.5 ng/ml) or TNF-α (200 ng/ml) plus IL-1β (0.5 ng/ml). After 24 h, they were transfected with 2 different Drak2 siRNAs (#592 (SEQ ID Nos:7 and 8) and #1162 (SEQ ID Nos: 5 and 6)), or with a control siRNA (SEQ ID Nos: 9 and 10), which had a scrambled sequence of siRNA #1162. Drak2 protein expression at 48 h was assayed by flow cytometry (FIG. 15A). Phospho-p70S6 kinase (upper panel) and total p70S6 kinase (lower panel) in the cell lysates were detected by immunoblotting (FIG. 15B). The ratios of phospho-p70S6 kinase versus total p70S6 kinase signals according to densitometry were expressed in a bar graph (FIG. 15C). D. Rapamycin protected cytokine-induced apoptosis in NIT cells. NIT-1 cells were stimulated with IFN-γ (1000 U/ml) plus IL-1β (0.5 ng/ml) or TNF-α (200 ng/ml) plus IL-1β (0.5 ng/ml) for 48 h in the presence or absence of rapamycin (250 nM). Their apoptosis was assessed by annexin V staining followed by flow cytometry.

FIG. 16—An alignment of the nucleic acid sequences of 3 Drak2 orthologs. The boxed sequences on the mouse sequence corresponds to the siRNAs used to inhibit Drak2 expression. The nucleotide identity is 85% between mouse and human, and 1005 between mouse and rat. The amino acid identity is 91% between mouse and human, and 100% between mouse and rat.

FIG. 17—An alignment of the nucleic acid sequences of 3 p70S6 kinase orthologs. The nucleotide identify is 95% between mouse and human, and 95% between mouse and rat. The amino acid identity is 99% between mouse and human, and 99% between mouse and rat.

FIG. 18—Inhibition of both the Drak2/p70S6kinase and mTORC1/p70S6kinase pathways shows additive protective effect on NIT-1 cells in apoptosis. Rapamycin and Drak2 siRNA showed additive protective effect on NIT-1 cells in apoptosis. NIT-1 insolinoma cells were treated with IFN-g+IL-1b for 72 hours, with or without 250 nM rapamycin. Drak2 siRNA was transfected to some cells 24 hours after initiation of the culture. Apoptosis of cells was measured with annexin-V staining followed by flow cytometry at 72 h.

FIG. 19—Drak2 siRNA (designed based on the mouse Drak2 sequence) effectively protects human islets from inflammatory cytokine-induced apoptosis. Human islets were treated with cytokines (IFN-γ (1000 U/ml), IL-1β (0.5 ng/ml), TNF-α (200 ng/ml), 24 h later, they were transfected with a combination of 2 Drak2 siRNA (#592 and #1162, 10 nM each). At 72 h, the islets were harvested, dispersed and tested for annexin V expression by flow cytometry. The percentage of apoptotic cells (annexin V positive) is shown.

### DESCRIPTION OF ILLUSTRATIVE EMBODIMENTS

The present invention has thus identified Drak2 as a critical member of the complex apoptotic pathway that is triggered in islet β-cell in TD1 and TD2. The identification of p70S6 kinase as a substrate of Drak2, further confirms the critical role played by the latter in molecular events leading to diabetes onset and development.

The present invention thus opens the way to diagnosis, therapeutic, and monitoring methods of both Type 1 and Type 2 diabetes. It also enables the set-up of screening assays to identify modulators of Drak2 level/activity. The screening assays of the present invention also enable the identification of therapeutics to treat or prevent diabetes onset or development.

Rapid Induction of Drak2 Expression in Islet β-Cells and its Association with Islet Apoptosis.

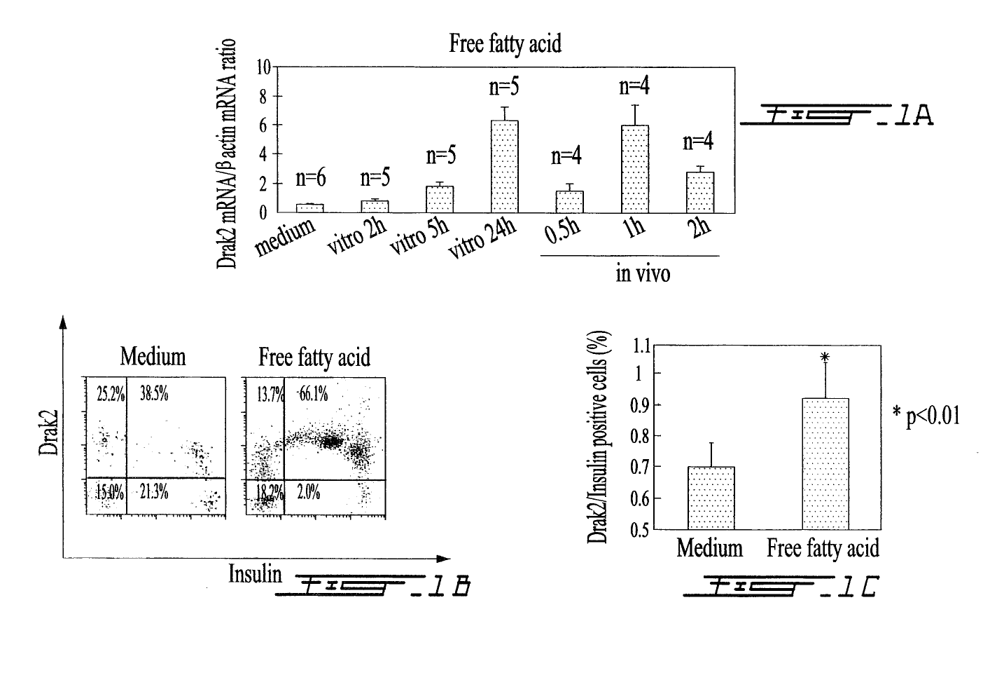

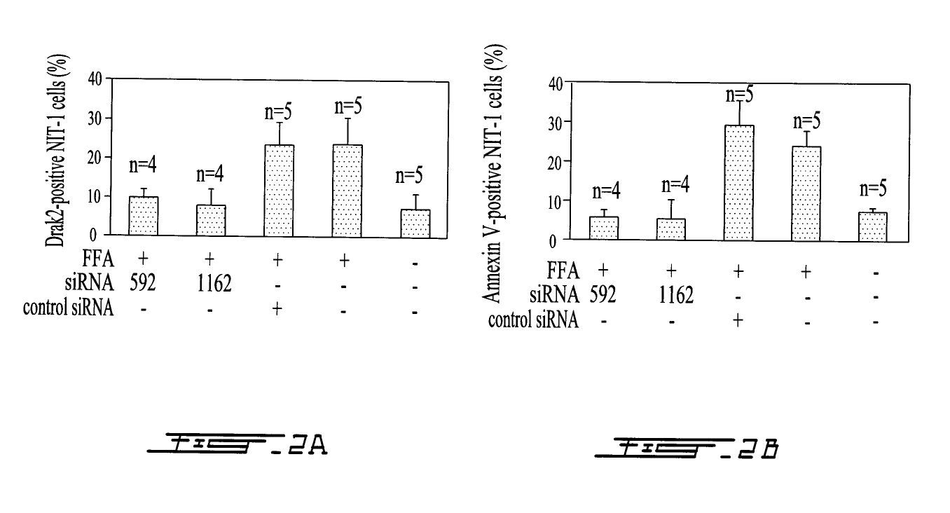

In T2D, high serum lipid is known to jeopardize islet function and survival (Ahren, B. 2005. Curr. Mol. Med. 5:275-286). When isolated islets were exposed to FFA in vitro or in vivo, Drak2 mRNA was drastically induced within 24 h and 1 h (from the time of FFA injection to mouse sacrifice; the time of islet isolation was not calculated in), respectively (FIG. 1A).

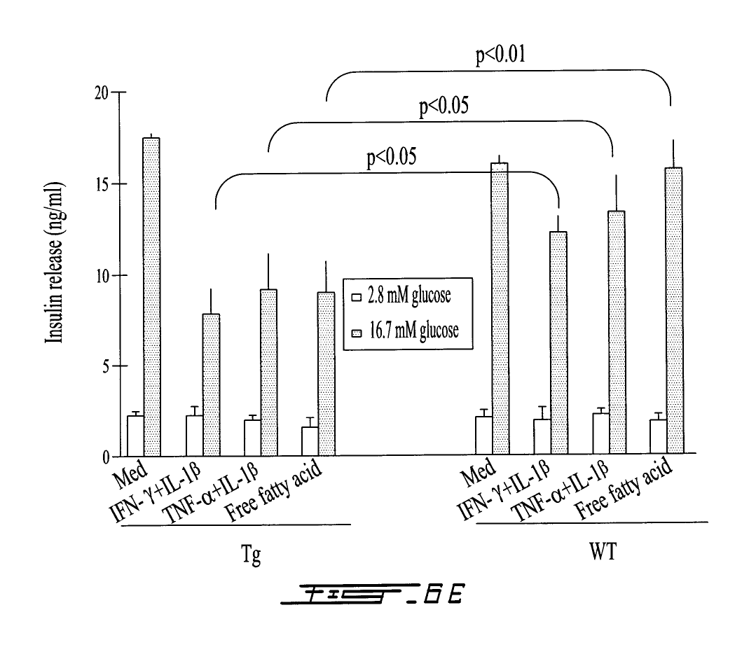

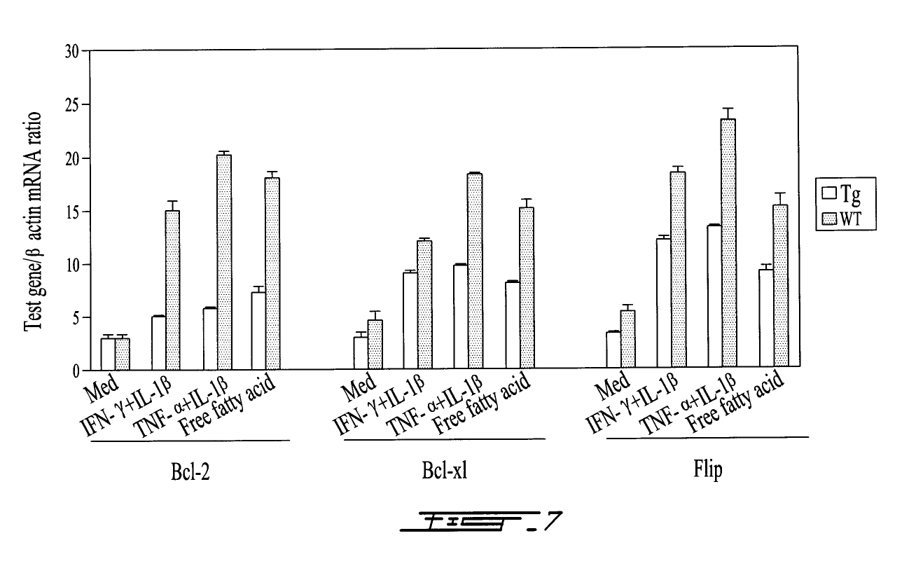

We next assessed Drak2 protein levels in β-cells, employing anti-insulin mAb and anti-Drak2 Ab in 2-color flow cytometry. When the islets were stimulated with FFA, Drak2 protein levels in insulin-positive β-cells were significantly augmented, as shown in histogram 1B; a summary of 3 independent experiments is illustrated in FIG. 1C. The finding on Drak2 protein increase was consistent with the heightened Drak2 mRNA expression. FFA, as expected, induced islet cell apoptosis (FIG. 4A, top row WT islets; FIG. 4B). Taken together, this data indicate that Drak2 overexpression in islets leads to their apoptosis.

Drak2 Knockdown by siRNA Protected NIT-1 Insulinoma Cells from FFA-Triggered Apoptosis.

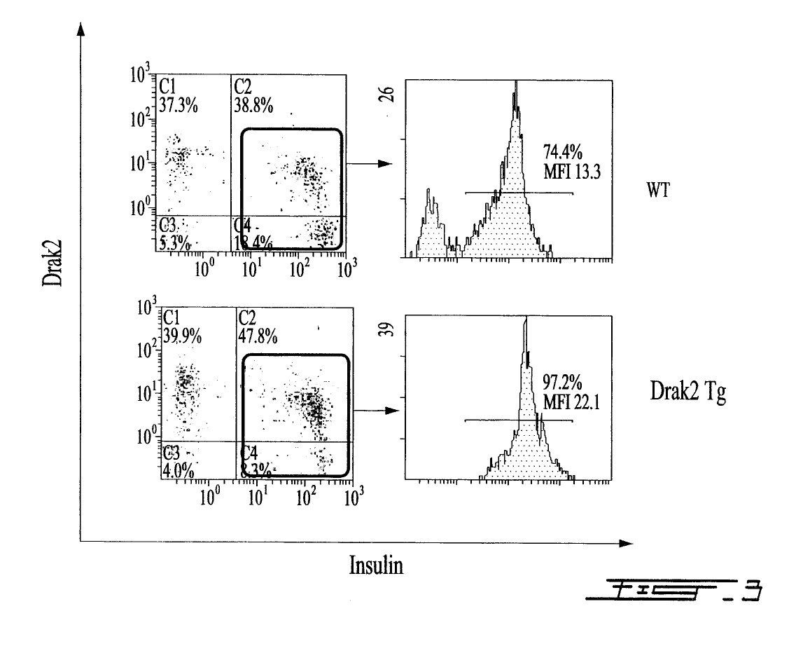

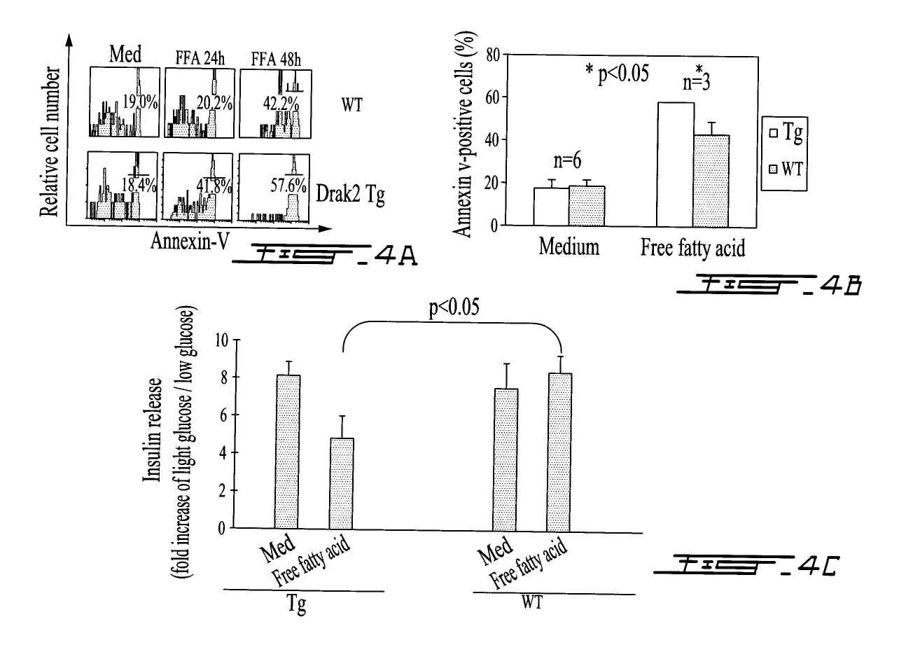

To prove that Drak2 was indeed critical to FFA-induced apoptosis, we employed as a Drak2 inhibitor, siRNA to prevent Drak2 upregulation in NIT-1 insulinoma cells. As shown in FIG. 2A, similarly to normal 1′-cells, Drak2 protein was induced in NIT-1 cells by FFA. Two different Drak2 siRNA significantly truncated Drak2 protein upregulation stimulated by FFA, but a control siRNA had no effect on the Drak2 level (FIG. 2B). As in normal islet cells, FFA induced NIT-1 cell apoptosis after 24 h. However, with protection by the 2 Drak2 siRNA, but not the control siRNA, such apoptosis induction was truncated (FIG. 2B).

Drak2 Overexpression in Tg Islets Aggravated FFA-Triggered Apoptosis

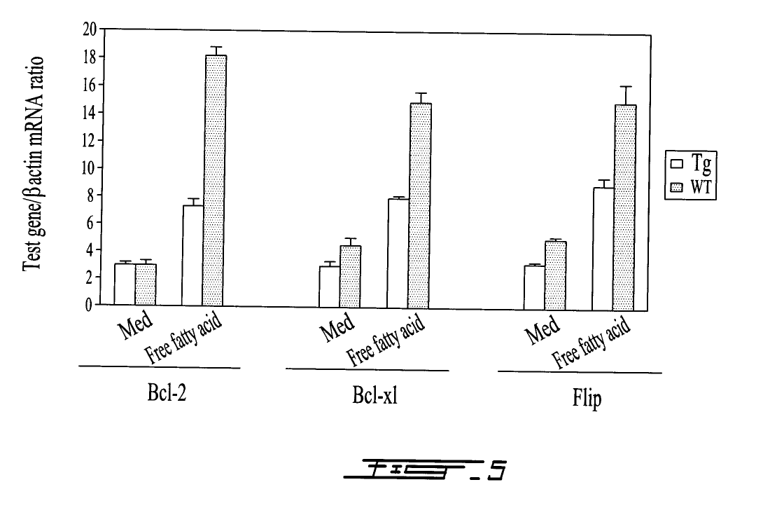

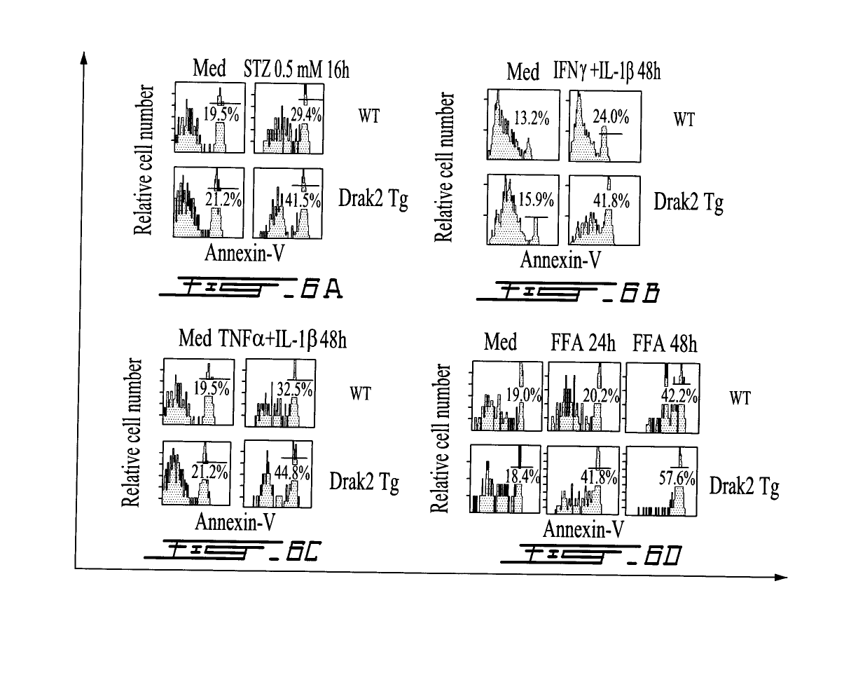

To further validate the role of Drak2 in islet survival, actin promoter-driven Drak2 Tg mice, as described in Mao et al., 2006. J. Biol. Chem. 281:12587-12595), were studied. These mice are viable, fertile, and have no gross anomalies. We demonstrated in FIG. 3 that Drak2 protein expression in insulin-positive Tg islet cells was augmented both in terms of mean fluorescent intensity and percentage of Drak2 positive cells, compared with wild type (WT) islet cells, according to Drak2/insulin two-colour flow cytometry.

When Tg islets were stimulated with FFA for 24 h, their apoptosis was significantly increased, as compared to WT islets (41.8% versus 20.2%, FIG. 4A; a summary of 3 experiments is illustrated in FIG. 4B). At 48 h, WT islets also started to suffer from apoptosis, but Tg islets were inflicted with more damage (FIG. 4A, 3rd column).

To pin-point the apoptotic cells in the islets as β-cells, and also to assess the function of β-cells, we evaluated islet insulin release after a 16.7 mM glucose stimulation. Insulin released by the Tg β-cells was significantly lower than by WT β-cells (FIG. 4C) after FFA assault. This confirmed that augmented Drak2 expression was harmful to β-cell survival and function.

Drak2 Overexpression Compromised Anti-Apoptotic Molecule Induction

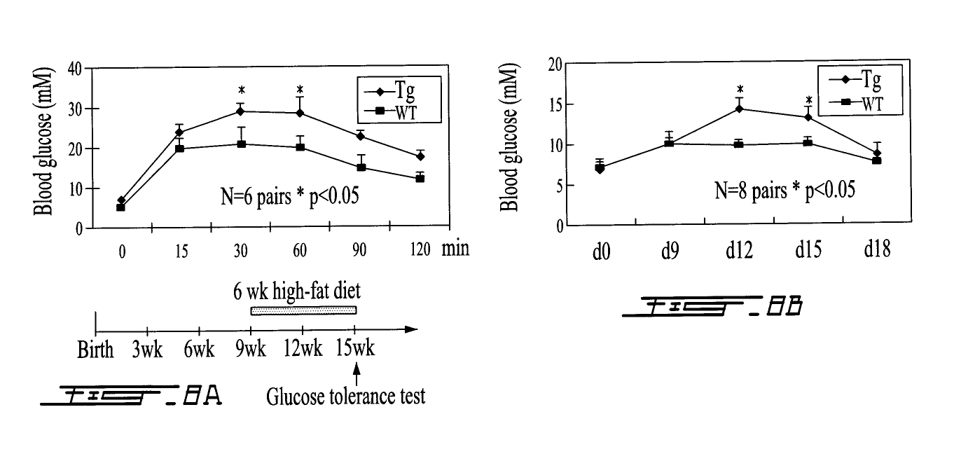

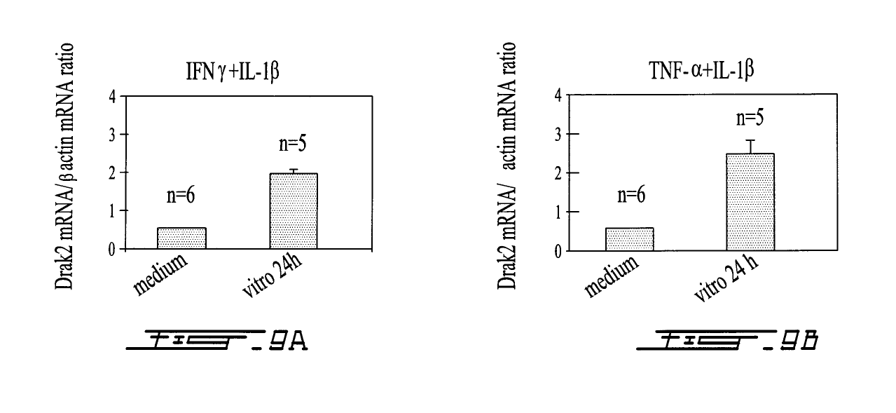

To understand the molecular mechanisms of β-cell apoptosis associated with Drak2 overexpression, we surveyed the expression levels of a group of anti-apoptotic factors in Tg versus WT β-cells. Anti-apoptotic factors Bcl-2, Bcl-xL and Flip were expressed at low levels in WT and Tg β-cells, but were significantly induced 24 h after FFA stimulation in WT β-cells (FIG. 5); but such induction was compromised in Tg β-cells. The data indicate that Drak2 overexpression in islets reduce the elevation of anti-apoptotic factors upon detrimental stimulation, and suggests that such compromise might be one of the reasons that renders β-cells to prone apoptose.

Drak2 Overexpression in Tg Islets Aggravated Cytokine- and FFA-Triggered Apoptosis

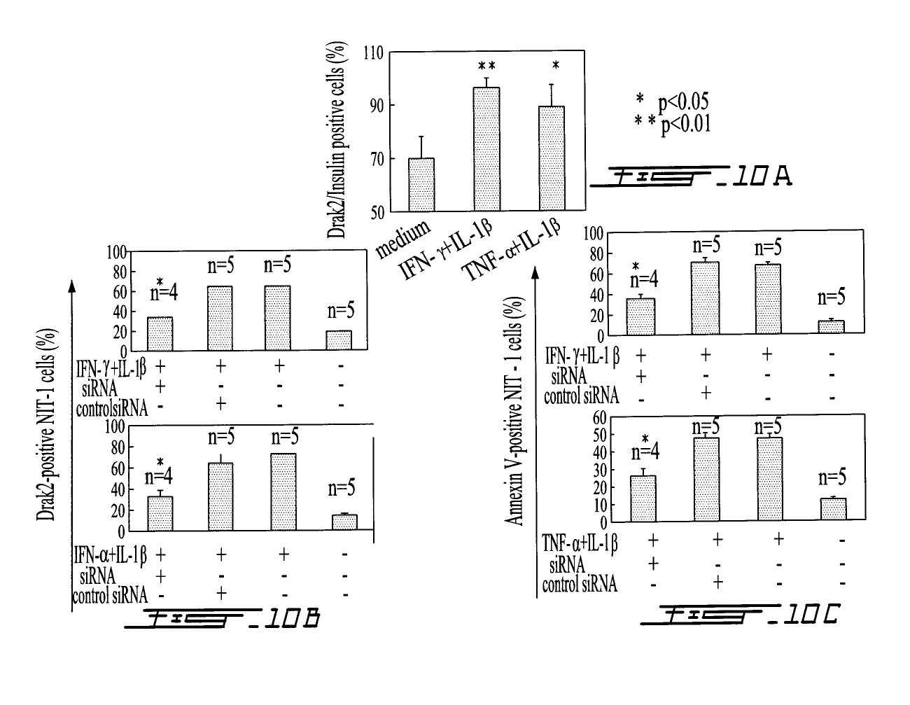

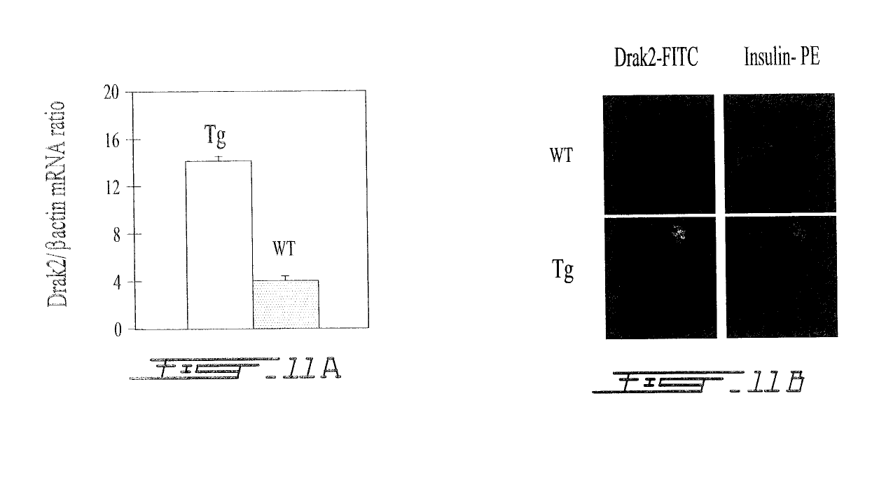

To further validate the role of Drak2 in islet survival, actin promoter-driven Drak2 Tg mice, as described in Mao et al., 2006 (Supra), were studied. These mice are viable, fertile, and have no gross anomalies. Drak2 mRNA was about 4 times higher in Tg islets as compared to WT islets. Immunofluorescence study revealed elevated Drak2 protein levels in Tg β-cells which were insulin-positive. When Tg islets were stimulated with STZ, IFN-γ plus IL-1β, TNF-α plus IL-1γ or FFA (FIG. 6A-D), their apoptosis was significantly increased, compared to WT islets. Insulin release assay demonstrated that β-cell function of Tg islets was significantly lower than in WT islets (FIG. 6E). These in vitro experiments confirmed that augmented Drak2 expression or increased activity was harmful to β-cell survival.

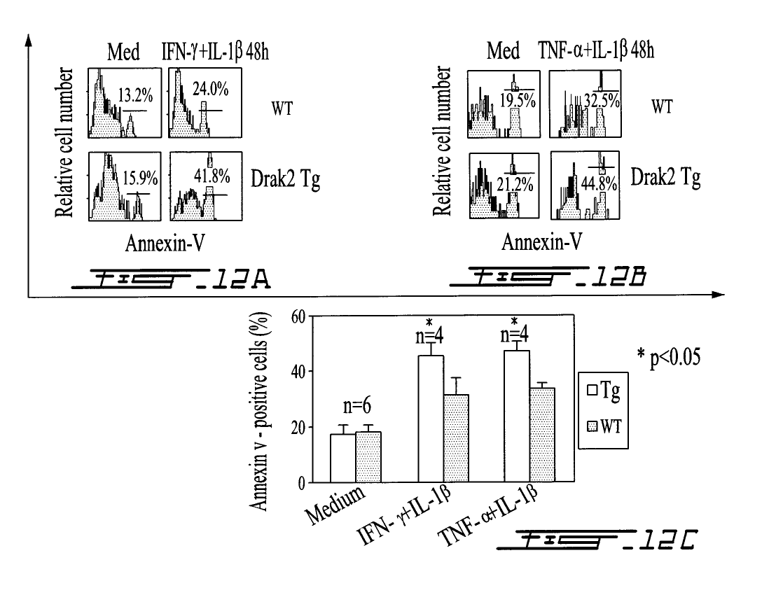

To understand the molecular mechanisms of β-cell apoptosis associated with Drak2 overexpression, we surveyed the expression levels of a group of anti-apoptotic factors in Tg versus WT β-cells. Anti-apoptotic factors Bcl-2, Bcl-xL and Flip were expressed at low levels in WT and Tg β-cells, but were significantly induced 24 h after IFN-γ plus IL-1β, TNF-α plus IL-1γ or FFA stimulation in WT β-cells (FIG. 7); however, such induction was compromised in Tg β-cells. The data suggest that Drak2 overexpression or increased activity in islets reduced the elevation of anti-apoptotic factors upon detrimental stimulation, and such compromise renders β-cells prone apoptotic.

Drak2 Overexpression Led to Increased T1D and T2D Risks In Vivo

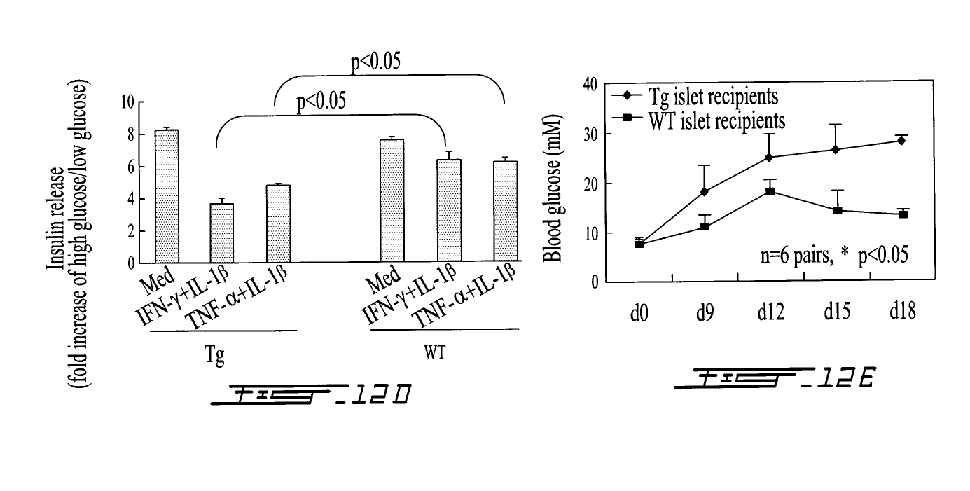

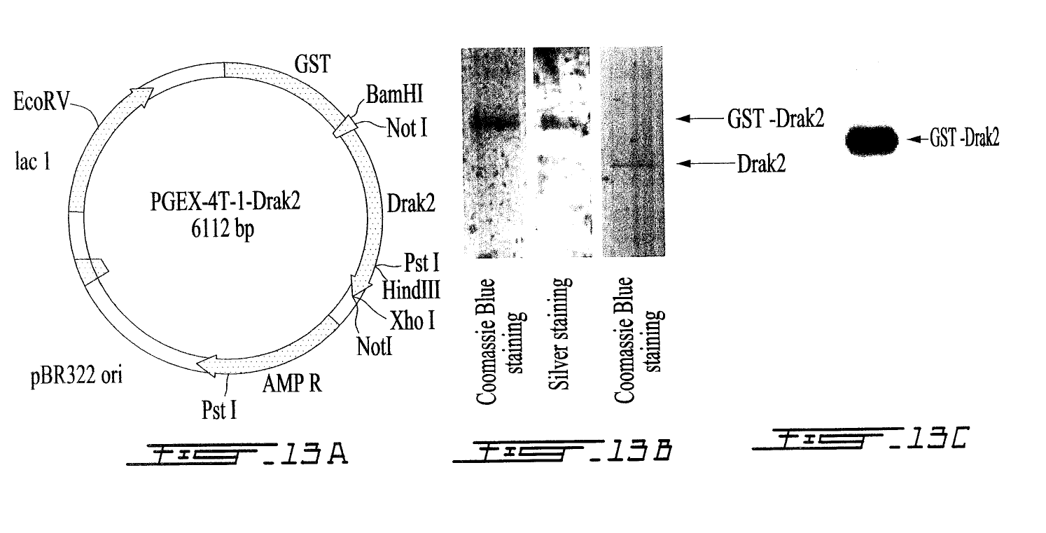

The proapoptotic effect of Drak2 in β-cells in vitro raised an intriguing question as to whether it was a diabetes risk gene. To assess this possibility, Drak2 Tg mice were subject to conditions mimicking T1D and T2D. For the former, Tg or WT mice were repeatedly injected with low doze STZ. According to previous reports, such treatments create a condition with chronic local inflammation in the pancreas similar to T1D. For this particular experiment, the STZ dose and injection frequency were adjusted so that most WT animals were at the borderline of overt diabetes, with blood glucose hovering around 10 mM. On days 12 and 15 after the initiation of STZ treatment, Drak2 Tg mice became overtly diabetic with blood glucose above 12 mM, and their levels were statistically significantly higher than those in WT mice (FIG. 8A). Thus, in combination with the in vitro data, these results suggest that augmented Drak2 expression or activity is a risk for T1D.

In T2D, islets also undergo apoptosis, due to assaults from inflammatory cytokines, as well as high blood glucose and lipid (Schutze 2004). We employed a diet-induced obesity model to simulate T2D (Winzell et al., 2004). Tg and WT mice in the C57BL/6 background at 9 weeks of age were fed a high fat-diet for 6 weeks. Both Tg and WT animals became overweight after this period, on average 10 g heavier than mice on a normal diet (data not shown). Both groups maintained normal fasting blood glucose levels. However, in the glucose tolerance test, Tg mice manifested statistically significantly higher blood glucose levels at 30, 60 and 90 min after glucose injection (FIG. 8B). This finding, along with our in vitro results on FFA, suggests that Drak2 overexpression or increased activity renders mice prone to T2D.

Rapid Induction of Drak2 Expression in Islet β-Cells and its Association with Islet Apoptosis

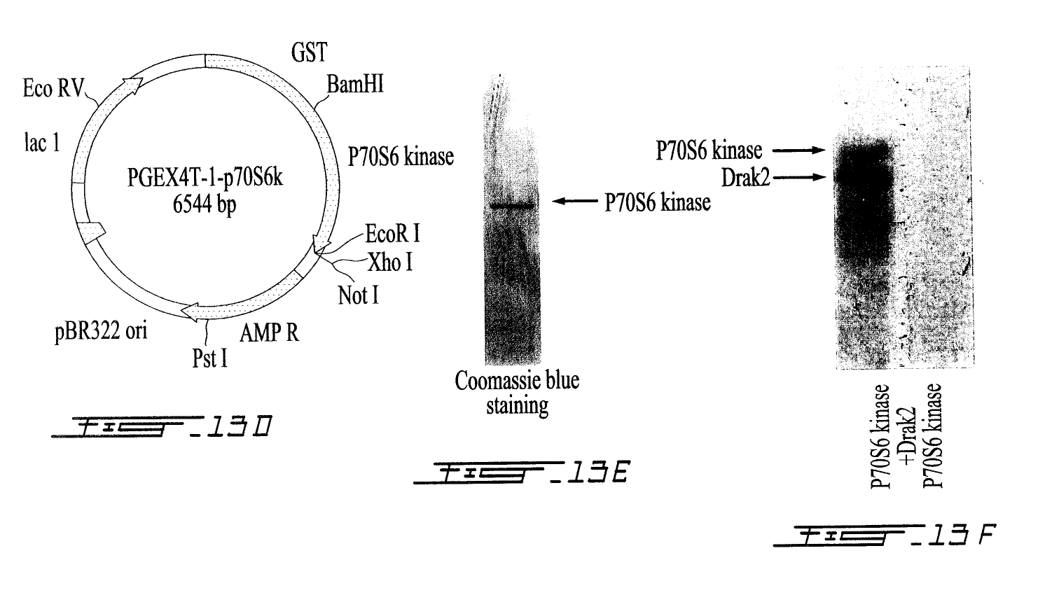

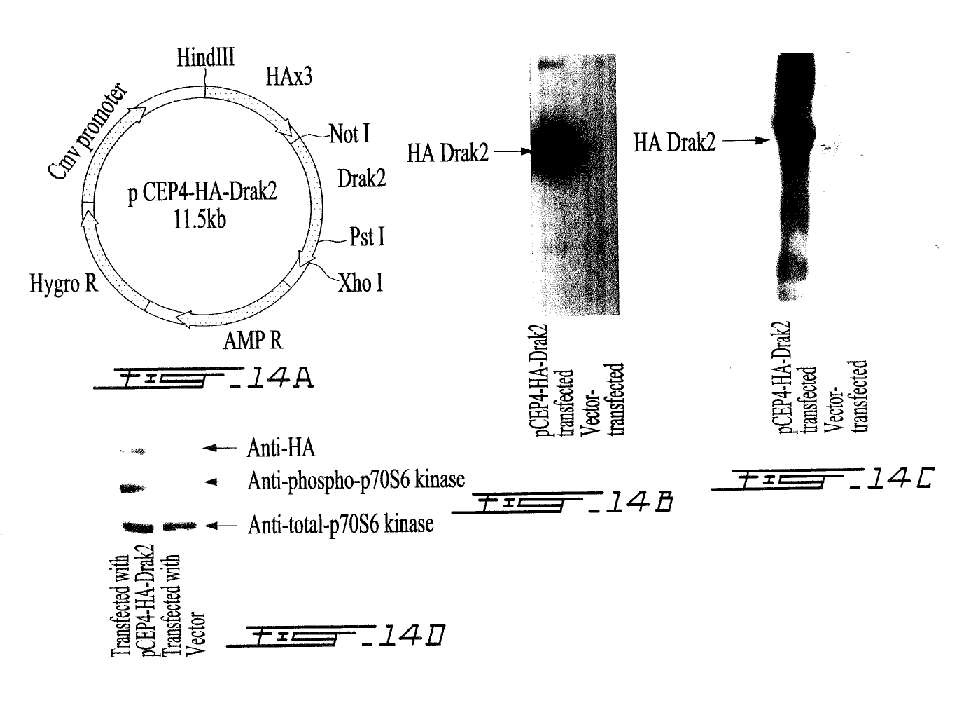

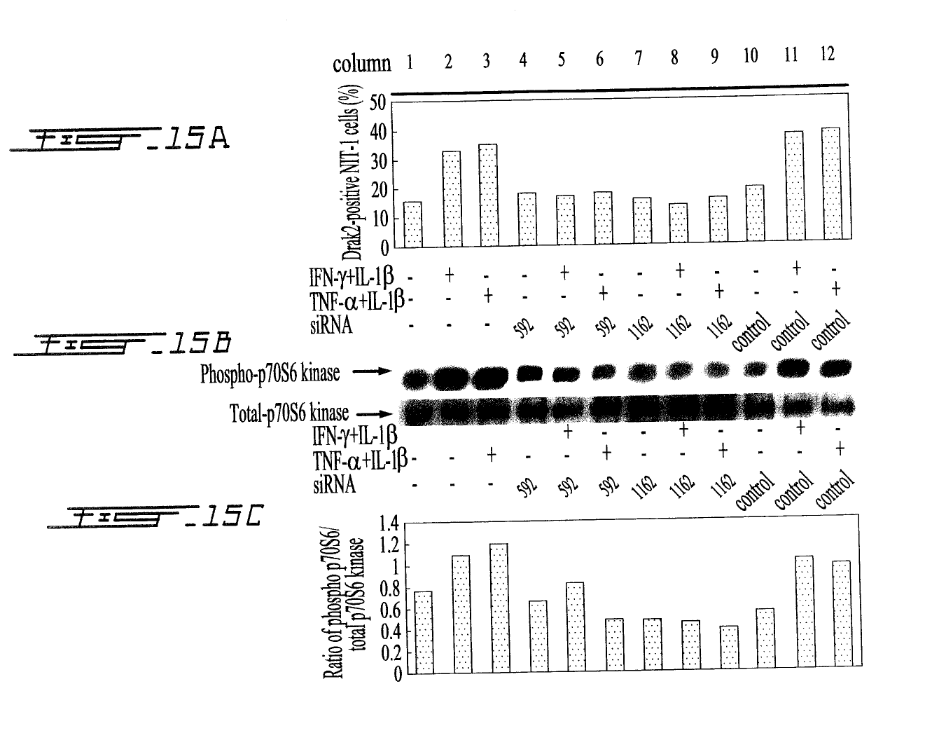

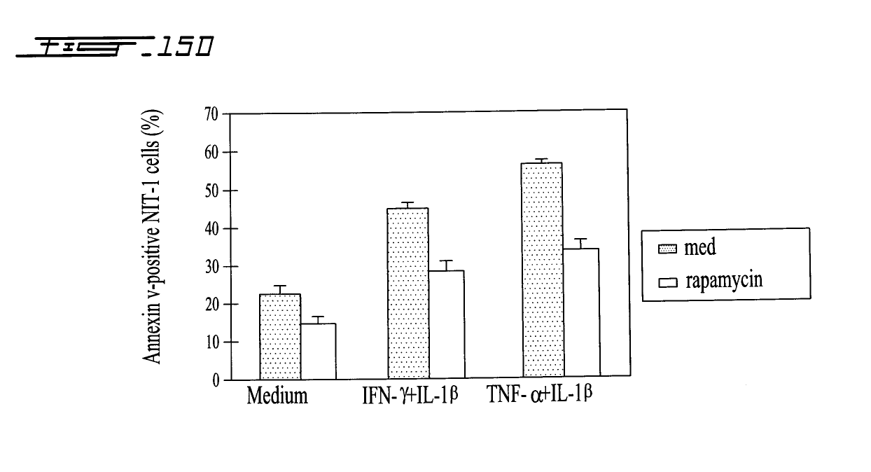

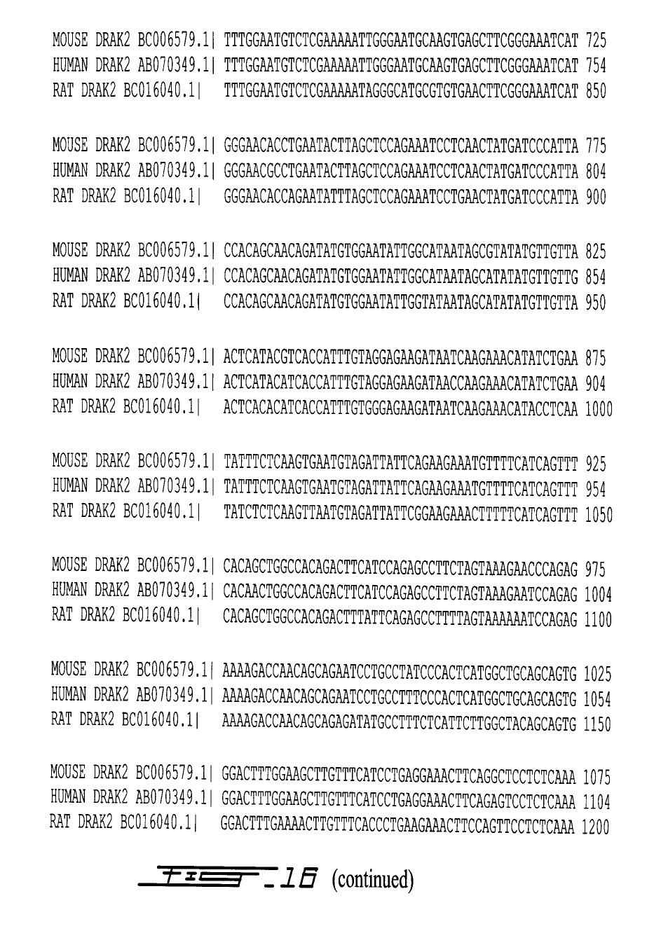

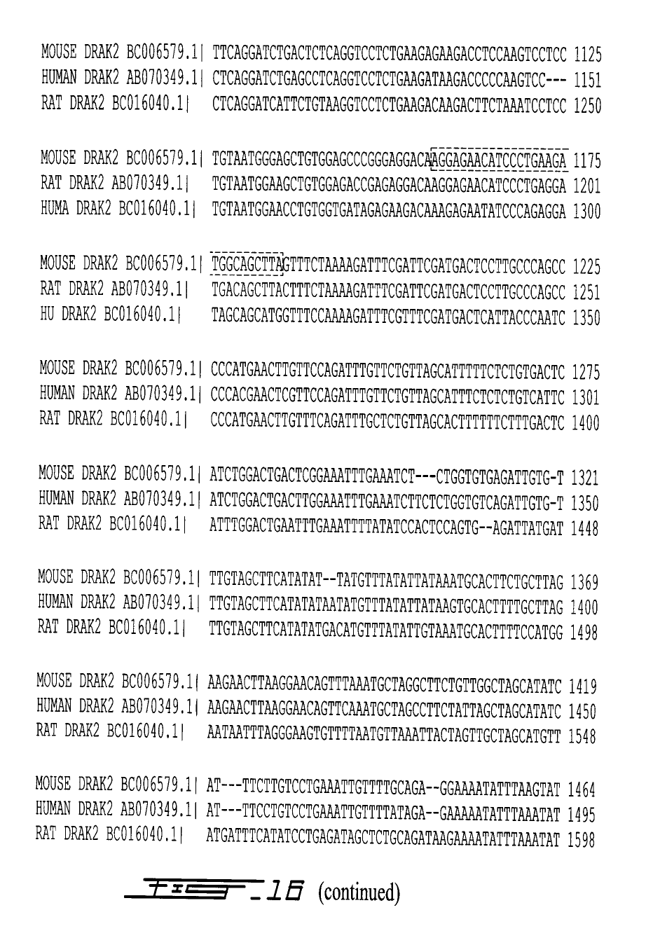

We treated islets with a combination of IFN-γ and IL-1β or TNF-α and IL-1β which are reported to cause islet apoptosis in type 1 diabetes (Aliza et al., 2006; Lee et al; 2004). These cytokines rapidly induced Drak2 mRNA expression in isolated islets within 24 h (FIGS. 9A and 9B). We next assessed Drak2 protein levels in β-cells employing anti-insulin mAb and anti-Drak2 Ab in 2-color flow cytometry. When the islets were stimulated with IFN-γ plus IL-1β, or TNF-α plus IL-1β. Drak2 protein levels in insulin-positive β-cells were significantly augmented, as shown in a summary of 4 independent experiments (FIG. 10A). This protein upregulation was consistent with the heightened Drak2 mRNA expression. These stimuli also induced islet cell apoptosis (FIGS. 12A and 12B, top rows; FIG. 12C, black columns, WT islets). Taken together, our data indicate that Drak2 overexpression in islets leads to islet cell apoptosis.

Drak2 Knockdown by siRNA Protected NIT-1 Insulinoma Cells from Cytokine-Triggered Apoptosis

To prove that Drak2 was indeed critical to cytokine-induced β-cell apoptosis, we employed siRNA to prevent Drak2 upregulation in NIT-1 insulinoma cells. As shown in FIG. 10B, similarly to normal β-cells, Drak2 protein was induced in NIT-1 cells by IFN-γ plus IL-1β 3rd bar, top panel) or TNF-α plus IL-1β 3rd bar, lower panel). siRNA #1162 prevented Drak2 protein upregulation stimulated by IFN-γ plus IL-1β and TNF-α plus IL-1β 1st bars, FIG. 10B). Control siRNA had no effect on Drak2 levels (2nd bars, FIG. 10B). As in normal islet cells, these stimuli induced NIT-1 cell apoptosis after 24 h (3rd bars, FIG. 10C). However, with protection by Drak2 siRNA (1st bars) but not control siRNA (2nd bars), such apoptosis induction by IFN-γ plus IL-1β (top panel) or TNF-α plus IL-1β (lower panel) was dampened (FIG. 10C). This result confirmed the detrimental role of Drak2 in islet β cell survival.

Transgenic Drak2 Overexpression in Tg Islets Aggravated Cytokine-Triggered Apoptosis

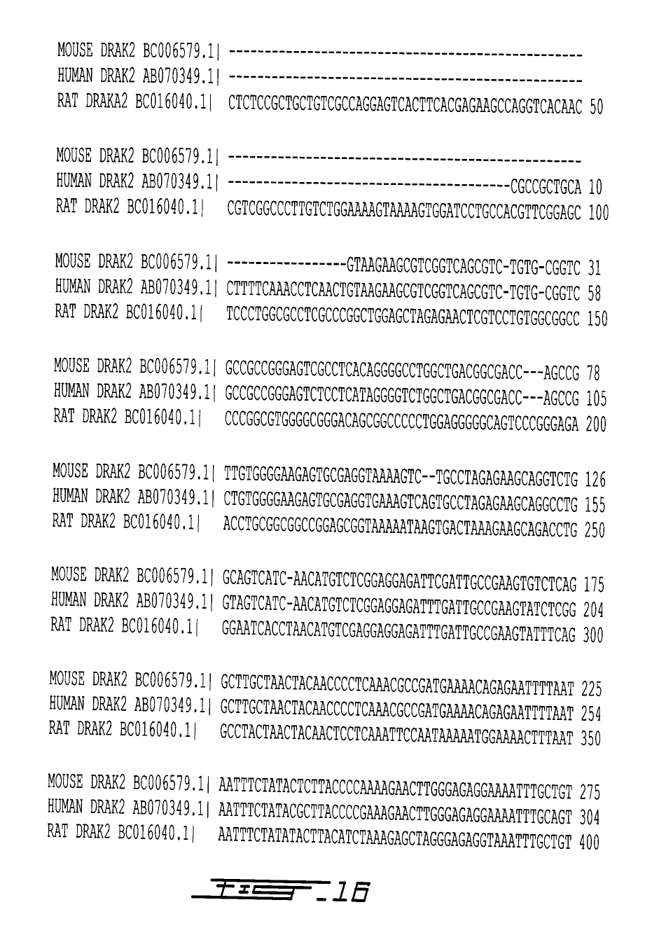

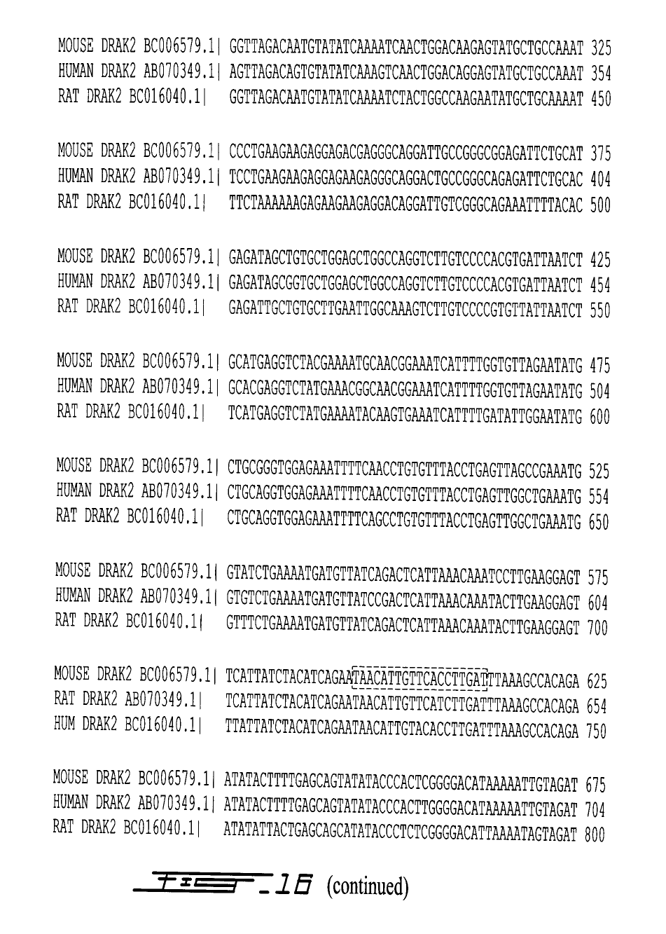

The role of Drak2 in islet survival was further validated using actin promoter-driven Drak2 Tg mice which we generated recently (Mao et al., 2006). These mice are viable, fertile, and have no gross anomalies (Mao et al., 2006). We demonstrated (FIG. 11A) that Drak2 mRNA was about 4 times higher in Tg islets than in WT islets. Immunofluorescence study revealed elevated Drak2 protein levels in insulin-positive Tg β-cells (FIG. 12B). Tg islet cells underwent increased apoptosis over WT islet cells when stimulated with IFN-γ plus IL-1β or TNF-α plus IL-1β (FIGS. 12A and 12B). A summary of data from 4-6 experiments are given in FIG. 12C. Insulin release assay demonstrated that the β-cell function of Tg islets assaulted by cytokines was significantly lower than that of WT islets (FIG. 12D), pinpointing the damage to β-cells. These in vitro experiments confirmed that augmented Drak2 expression was harmful to β-cell survival.

Drak2 Overexpression Led to Increased Type 1 Diabetes Incidence In Vivo

The proapoptotic effect of Drak2 in β-cells in vitro raised a logical question as to whether its overexpression would render mice prone to type 1 diabetes. In our Tg mice, Drak2 expression was not restricted to islets as it was driven by the actin promoter (Mao et al., 2006). To pin-point the in vivo phenotype to islets, we transplanted Tg or WT islets to full-dose STZ (200 mg/kg)-induced diabetic mice, which were syngeneic to the donors. Once the recipients became normoglycemic, glucose tolerance tests were performed to ascertain that they had similar reserve islet capacity (data not shown). These recipients were then injected with multiple low-doses of STZ to induce borderline diabetes in WT islet recipients. Islet damage by such a STZ regimen is reported to mimic that in type 1 diabetes (Liadis et al., 2005; Pighin et al., 2005). After STZ injection, the blood glucose levels of WT mice hovered around 12 mM (FIG. 12E). However, such treatment caused full-blown diabetes in Tg islet recipients from days 15 to 18 post STZ treatment (FIG. 12E), with their blood glucose rising above 20 mM. This finding clearly indicates that Drak2 overexpressed in Tg islets is responsible for the type 1 diabetes-prone phenotype in the recipients.

Identification of p70S6 Kinase as a Drak2 Substrate In Vitro

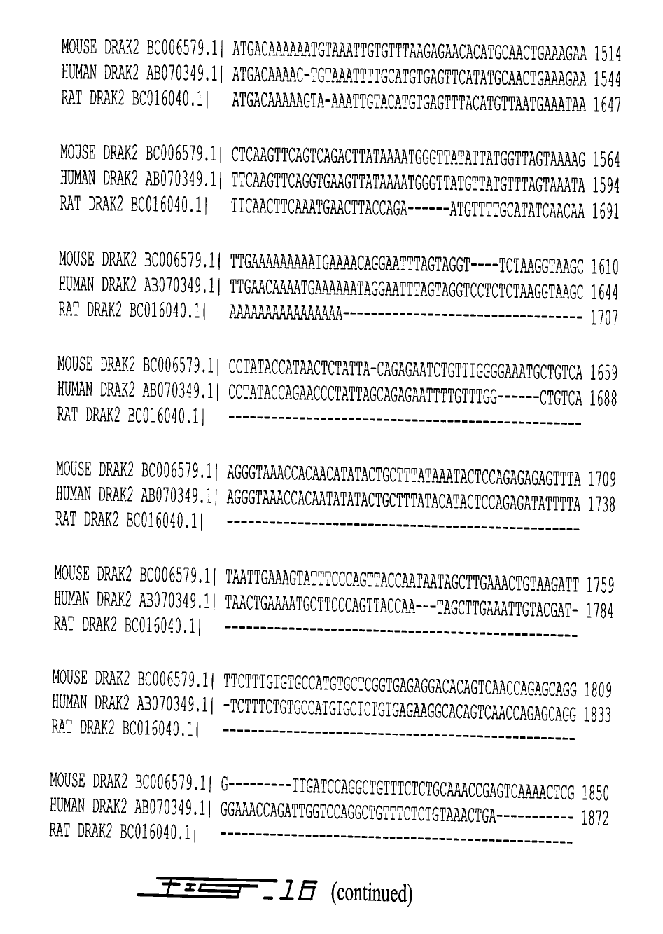

To understand the mechanism of Drak2 action, we attempted to discover the substrate of Drak2. Recombinant mouse GST-Drak2 was generated with the construct pGEX-4T-1-Drak2 (FIG. 13A), and was prepared to more than 95% purity after size fractionation followed by affinity purification (left lane, FIG. 13B). Its kinase activity was confirmed by autophosphorylation in an in vitro kinase assay (FIG. 13C). It was then employed as the kinase in an assay with the Invitrogen Protoarray Kinase Substrate Identification Kit, which contained 5,000 potential kinase substrate proteins of human origin. Five proteins showed a Z-score above 3, a threshold indicating more that 99.9% confidence. Among the 5 proteins, one was p70S6 kinase.

To confirm that mouse Drak2 could phosphorylate mouse p70S6 kinase, GST-tagged mouse p70S6 kinase was generated with the construct pGEX-4T-1-p70S6K (FIG. 13D), and processed to more that 95% purity after affinity purification followed by cleavage of GST by thrombin (FIG. 13E). Mouse Drak2, which was also more than 95% pure (right lane, FIG. 13B,) after affinity purification followed by cleavage of GST by thrombin, served as a kinase in an in vitro kinase assay, using mouse p70S6 kinase as a substrate. As illustrated in FIG. 12F, Drak2 could autophosphorylate itself, as expected (lane 1). It also phosphorylated mouse p70S6 kinase (lane 1). On the other hand, p70S6 kinase could not autophosphorylate (lane 2) in the kinase assay. Thus, the phosphorylation on mouse p70S6 kinase was caused by mouse Drak2, and p70S6 kinase was a bona fide Drak2 substrate in vitro.

Identification of p70S6 Kinase as a Drak2 Substrate In Vivo

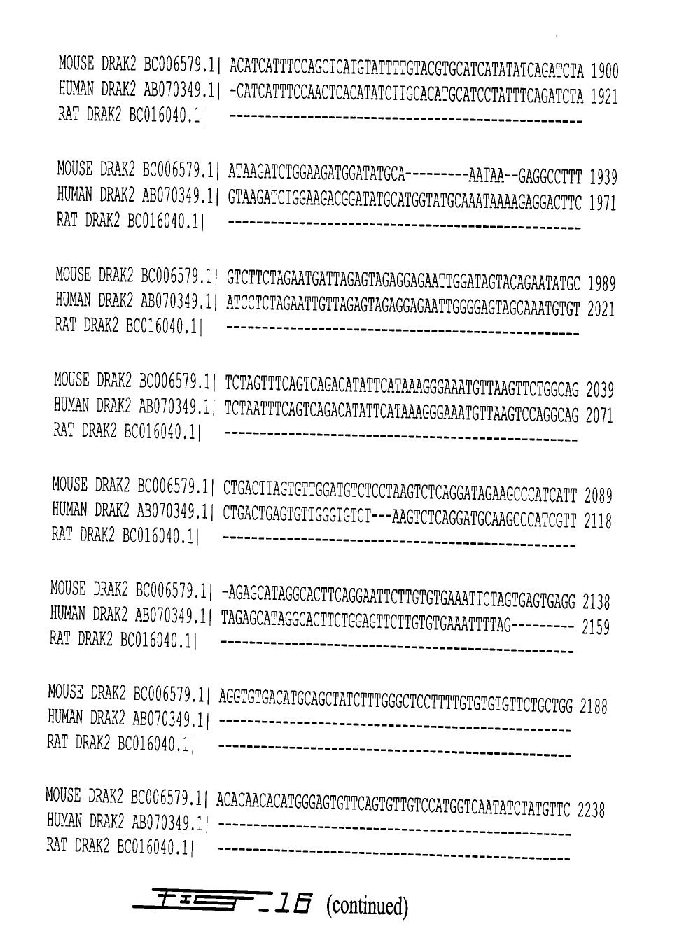

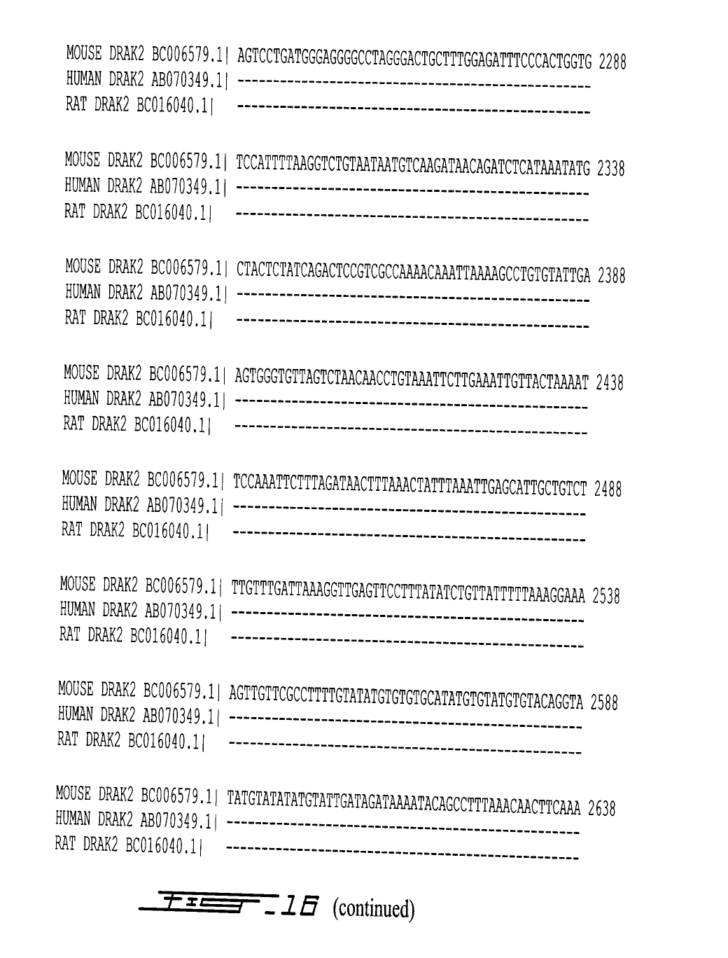

Next, we attempted to demonstrate that p70S6 kinase was a Drak2 substrate in vivo. NIT-1 cells were transiently transfected with a HA-tagged Drak2 expression construct pCEP-HA-Drak2 (FIG. 14A). HA-tagged Drak2 was affinity-purified, and it showed the expected size in immunoblotting (FIG. 14B). It was tested in an in vitro kinase assay and could autophosphorylate itself, as illustrated in FIG. 14C, proving that the recombinant protein possessed active kinase activity. When NIT-1 cells were transiently transfected with pCEP-HA-Drak2 or an empty vector, recombinant HA-Drak2 expression at the size of 45 kD could be detected by anti-HA Ab in immunoblotting in the former but not in the latter transfected cells, as seen in FIG. 14D (lane 1 versus lane 2, top panel). In pCEP-HA-Drak2-transfected cells (lane 1, middle panel, FIG. 14D) but not empty vector-transfected cells (lane 2, middle panel, FIG. 14D), p70S6 kinase phosphorylation was augmented, while total p70S6 kinase protein remained constant (bottom panel, FIG. 14D). This indicates that Drak2 overexpression in vivo led to increased p70S6 kinase phosphorylation, and corroborates our in vitro data that p70S6 kinase was a Drak2 substrate.

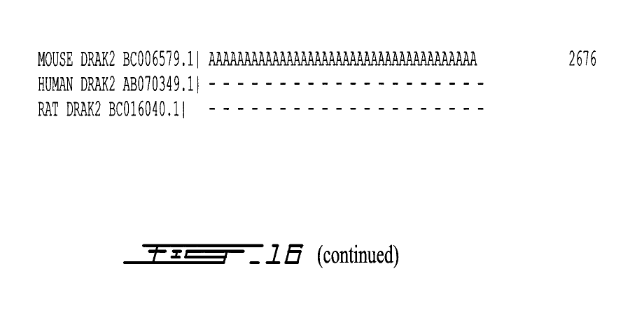

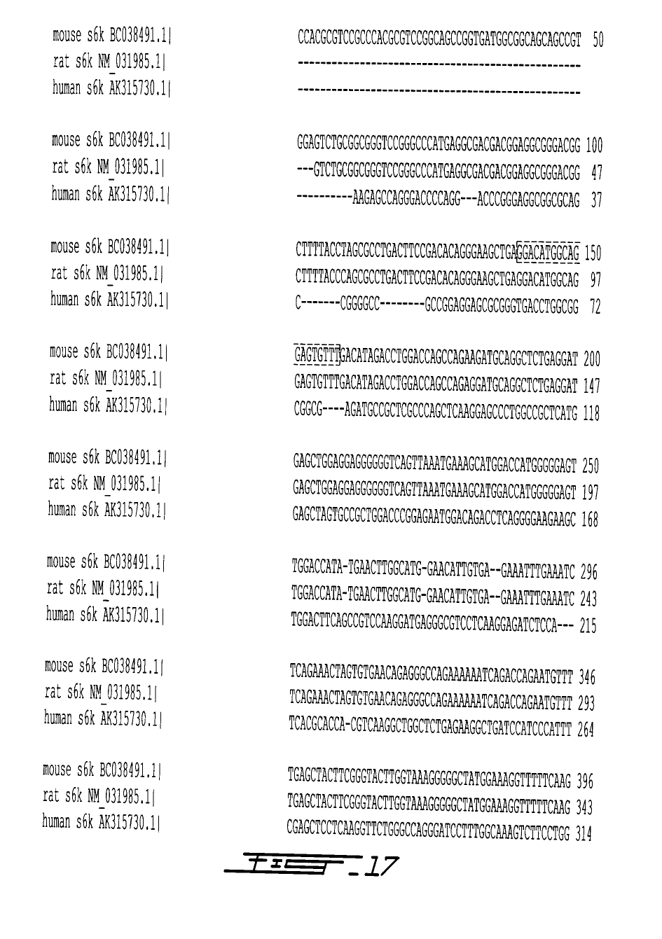

Further in vivo verification of the relationship between Drak2 and p70S6 kinase phosphorylation was undertaken by knocking down Drak2 expression with siRNA. As depicted in FIG. 15, IFN-γ plus IL-113 or TNF-α plus IL-1β induced Drak2 protein expression (the 2nd and 3rd columns, compared with the 1st column; FIG. 15A). This was accompanied by increased p70S6 kinase phosphorylation (the 2nd and 3rd lanes, compared with the 1st lane, FIG. 15B; the 2nd and 3rd columns, compared with the 1st column, FIG. 15C). Control siRNA had no effect on Drak2 induction (the last 2 columns compared with the 2nd and 3rd columns, FIG. 15A), nor did it on p70S6 kinase phosphorylation (the last 2 lanes compared with the 2nd and 3rd lanes, FIG. 15B; last the 2 columns compared with the 2nd and 3rd columns, FIG. 15C). However, 2 different Drak2 siRNAs knocked down cytokine-induced Drak2 expression (columns 5, 6, 8, and 9, compared with columns 2 and 3, FIG. 15A), and this was accompanied by reduced cytokine-induced p70S6 kinase phosphorylation (lanes 5, 6, 8 and 9, compared with lanes 2 and 3, FIG. 15B; columns 5, 6, 8, and 9, compared with columns 2 and 3, FIG. 15C). This further confirms that p70S6 kinase was a Drak2 substrate in vivo.

To study the relevance of p70S6 kinase in β-cell apoptosis, we used rapamycin to inhibit mTORC1, which is another kinase capable of phosphorylating p70S6 kinase. NIT-1 cells under rapamycin protection showed reduced apoptosis upon inflammatory cytokine exposure (FIG. 15D), revealing that p70S6 kinase activity was indeed relevant top-cell apoptosis.

Inhibition of Both the Drak2/p70S6Kinase and mTORC1/p70S6Kinase Pathways Shows Additive Protective Effect on NIT-1 Cells in Apoptosis.

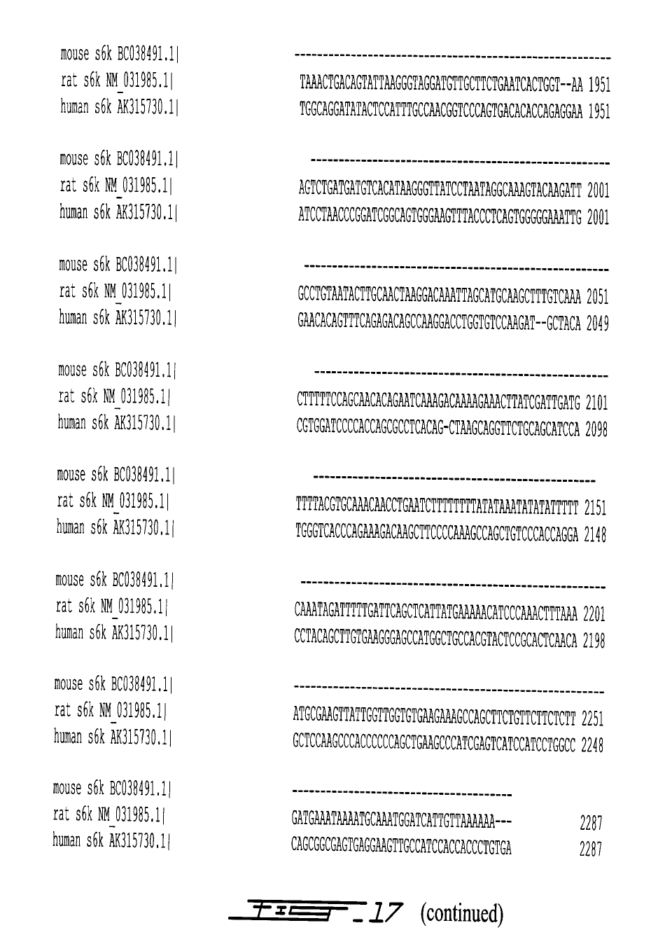

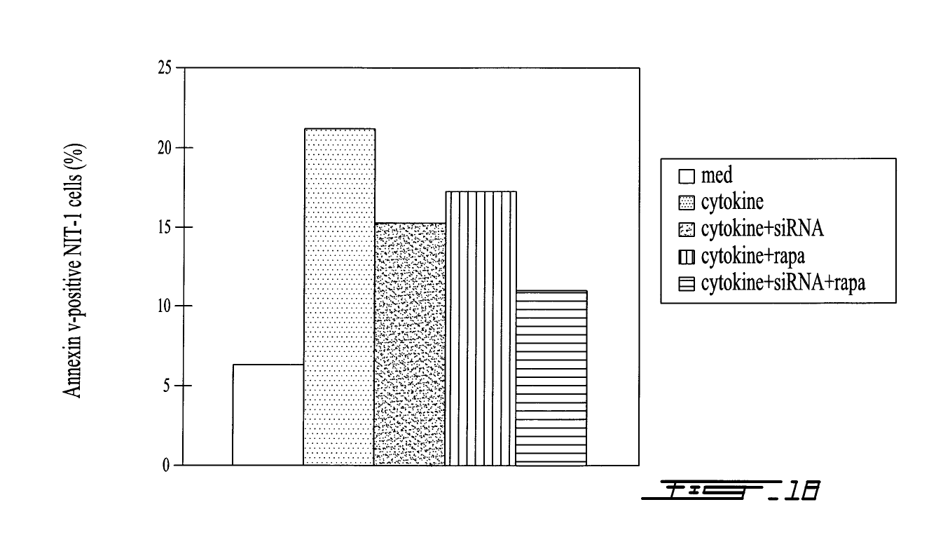

Since rapamycin and Drak2 siRNA could both individually inhibit p70S6K phosphorylation via two different pathways, which seem to be both activated during cytokine-induced β-cell apoptosis, we inquired as to whether the effect of rapamycin and Drak2 siRNA might be additive. To test this possibility, we treated NIT-1 cells with inflammatory cytokines IFN-γ+IL-1β to induce their apoptosis, and added raramycin and Drak2 siRNA individually or in combination to protect them from apoptosis. As shown in FIG. 18, rapamycin and Drak2 siRNA alone could reduce apoptosis, as expected, from the prior results. Of interest, however was that the combination of the two yielded better protective effect. These data strongly suggest that a strategy of combined use of an S6 kinase inhibitor (i.e., a mTORC1/p70S6kinase pathway inhibitor, such as rapamycin) plus Drak2 inhibitors will have better islet protecting effect. Such combinations could prevent or delay the onset of both type I and type II diabetes, as islet death plays a pivotal role in both diseases, albeit at different stages.

Drak2 siRNA (Designed Based on the Mouse Drak2 Sequence) Effectively Protects Human Islets from Inflammatory Cytokine-Induced Apoptosis.

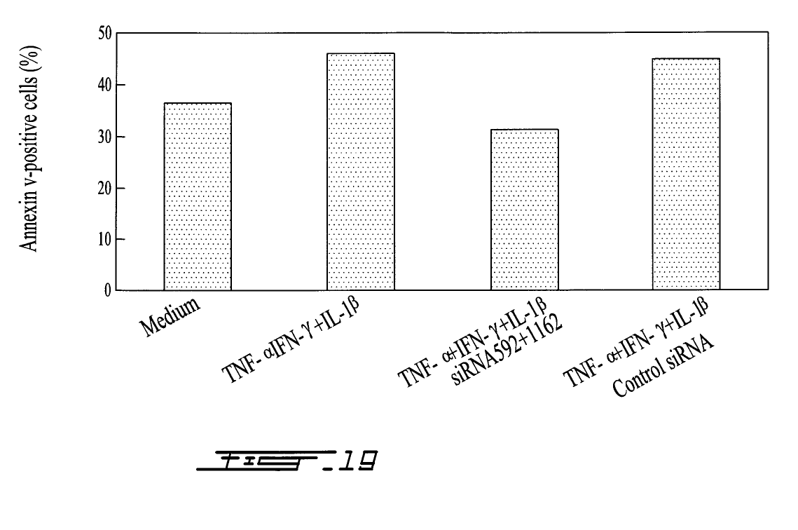

To prove that the data herein presented were translatable to the human situation, and not limited to mouse, the siRNAs designed from the mouse Drak2 sequence were used on islet cells isolated from patients. The data shown in FIG. 19, clearly shows that the approach of the present invention, applicable both in vitro and in vivo in mice predict their effectiveness in humans. The present invention thus opens the way to diagnosis and treatment of diabetes in humans.

The present invention is illustrated in further details by the following non-limiting examples.

### Example 1

### Islet Purification

Islet purification is performed as we described before (Wu et al., 2003 and 2004). Briefly, 2-ml of digestion solution (Hanks' balanced salt solution [HBSS] containing 20 mM HEPES and 2 mg/ml collagenase IV (Worthington Biochemical, Lakewood, N.J.) were injected into the common bile duct of Tg or wild type (WT) mice (20-24 g) after the distal end of the duct was ligated. The distended pancreas was isolated and put into a 15-ml tube containing an additional 0.5 ml of digestion solution. The pancreas was digested at 370 C for exactly 28 min, and the digestion process was stopped by the addition of 10 ml of cold HBSS containing 20 mM HEPES. The islet suspension was filtered through No. 7880 cheesecloth gauze (Tyco Healthcare, Mansfield, Mass.) and centrifuged at 500 g for 1-2 min. The pellet was washed with cold HBSS once at 500 g for 1-2 min, and the supernatant was removed completely. The pellet was then resuspended in 3 ml of 25% Ficoll, and 2-ml layers of 23, 20, and 11% Ficoll were added sequentially. The Ficoll gradient was centrifuged at 700 g for 5 min. Most of the islets were in the interface between the 20 and 23% Ficoll layers and were handpicked with Pasteur pipettes. They were then washed twice with cold HBSS. The islets were cultured overnight in RPMI 1640 containing 10% FCS, and then used for experimentation.

### Example 2

### Real Time RT-PCR

Drak2, Bcl2, Bcl-xL and Flip mRNA in islets was measured by real time RT-PCR as described in our previous publication (Mao et al., 2006).

### Example 3

### Flow Cytometry

Drak2 Tg and WT islets were digested with 0.05% trypsin-EDTA to obtain single cell suspensions. The cells were fixed with 4% paraformaldehyde and permeabilized with 0.2% Triton X-100. They were stained with rabbit anti-Drak2 Ab (Abgent, San Diego, Calif.; 1:50 dilution) and anti-insulin mAb (Sigma, St. Louis, Mo.; 1:500 dilution). Subsequently, they were stained with FITC-conjugated sheep anti-rabbit antibody (Chemicon, Temecula, Calif.), and PE-conjugated goat anti-mouse antibody (Jackson Immunoresearch, West Grove, Pa.), and analyzed by 2-color flow cytometry. Dispersed islet cells or small interfering RNA (siRNA)-transfected NIT-1 cells were also analyzed for apoptosis by flow cytometry using FITC-annexin V staining (Murakami et al., 2004).

### Example 4

### Drak2 Knockdown by siRNA in NIT-1 Cells

NIT-1 cells, derived from mouse insulinoma, were transfected with siRNA using Lipofectamine 2000 (Invitrogen, Burlington, Ontario) according to the manufacturer's instructions. For Drak2 siRNA, the oligonucleotide RNA sequences were CAUCCCUGAAGAUGGCAGCtt and GCUGCCAUCUUCAGGGAUGtt. The control was the scrambled sequence of said siRNA with following sequences: 5′CCCUAAGUGUAGGACGCACtt and 3′GUGCGUCCUACACUUAGGGtt. Single stranded RNA pairs were annealed by being incubated for 1 min at 900 C, and then cooled down to room temperature over 45 min. The final concentration of double-stranded siRNA was 20 μM for transfection.

### Example 5

### Insulin Release Assay

After 48 h culture in complete F-12K medium with 10% FCS in the absence or presence of various stimulants, the islets were transferred to 12-well plates at a density of 10 islets/well. The islets were gently washed twice with 1 ml Kreb's buffer (NaCl, 135 mM; KCl, 3.6 mM; NaH2PO4, 5 mM; MgCl2, 0.5 mM; CaCl2, 1.5 mM; NaHCO3, 2 mM; HEPES, pH 7.4, 10 mM; BSA, 0.07%), and then incubated in Kreb's buffer containing 2.8 mM glucose for 5 min at 370 C. Two hundred micro litres of supernatant were removed for determination of basal insulin levels. The islets were cultured for additional 40 min, and all the supernatants were harvested for determination of insulin levels as 2.8 mM glucose-stimulated release. The islets were then cultured in Kreb's buffer containing 16.7 mM glucose for 45 min at 370 C, and the supernatants were harvested for determination of insulin levels as 16.7 mM glucose-stimulated release. The insulin was assayed by ELISA (Linco Research, St. Charles, Mo.). The basal insulin levels, which were near zero, were deducted from the 2.8 mM and 16.7 mM glucose-stimulated levels in final data presentation.

### Example 6

### Glucose Tolerance Tests

Tg and WT mice were fed a high-fat diet (45% of total calories in the form of fat; Research Diets Inc. New Brunswick, N.J.) from age 9 weeks for 6 weeks. They were then fasted for 16 h and injected i.p. with D-glucose (2 mg/g body weight) in PBS. Blood samples from the tail vein were taken at 15, 30, 60, 90, and 120 min after injection for glucose measurements with a glucose meter (Bayer, Toronto, Ontario).

### Example 7

### Flow Cytometry (FIGS. 9-15)

Dispersed islet cells were fixed with 4% paraformaldehyde and permeabilized with 0.2% Triton X-100. For Drak2 and insulin detection, the cells were stained as described for confocal microscopy, and analyzed by 2-color flow cytometry. Dispersed islet cells or small interfering RNA (siRNA)-transfected NIT-1 cells were also analyzed for apoptosis by flow cytometry with FITC-annexin V staining (Murakami et al., 2004)).

### Example 8

### Drak2 Knockdown by siRNA in NIT-1 Cells (FIGS. 9-15)

NIT-1 cells, derived from mouse insulinoma, were transfected with siRNA using Lipofectamine 2000 (Invitrogen, Burlington, Ontario) according to the manufacturer's instructions. Two siRNAs specific for Drak2 were employed. For Drak2 siRNA #1162, the oligonucleotide RNA sequences were CAUCCCUGAAGAUGGCAGCtt and GCUGCCAUCUUCAGGGAUGtt. For Drak2 siRNA #592, the oligonucleotide RNA sequences were UAACAUUGUUCACCUUGAUtt and AUCAAGGUGAACAAUGUUAtt. The control siRNA was the scrambled sequence of siRNA #1162 with the following sequences: 5′CCCUAAGUGUAGGACGCACtt and 3′GUGCGUCCUACACUUAGGGtt. Single-stranded RNA pairs were annealed by incubation for 1 min at 900 C, and then cooled down to room temperature over 45 min. The final concentration of double-stranded siRNA was 10 nM for transfection.

### Example 9

### Confocal Microscopy

Drak2 Tg and WT islets were digested with 0.05% trypsin-EDTA to obtain single cell suspensions. The cells were placed on slides by Cytospin (Shandon, Pittsburgh, Pa.), fixed with 4% paraformaldehyde and permeabilized with 0.2% Triton X-100. The slides were stained with rabbit anti-Drak2 Ab (Abgent, San Diego, Calif.; 1:50 dilution) and anti-insulin mAb (Sigma, St. Louis, Mo.; 1:500 dilution). Subsequently, the slides were stained with FITC-conjugated sheep anti-rabbit antibody (Ab) (Chemicon, Temecula, Calif.), and PE-conjugated goat anti-mouse antibody (Jackson Immunoresearch, West Grove, Pa.). The cells were visualized under a Carl Zeiss confocal microscope, with excitation at 488 nm and emission at 505-550 nm for FITC, and with excitation at 543 nm and emission at 560-615 nm for PE. Intracellular Drak2 is shown in green, and intracellular insulin is in red.

### Example 10

### Islet Transplantation

Diabetes was induced in C57BL/6 mice by streptozocin (STZ) (200 mg/kg body weight, i.p.). After 14 days, syngeneic Tg or WT islets were transplanted into the peritoneal cavity of these diabetic mice (400 islets per mouse) to render the recipients euglycemic. Two weeks after islet transplantation, glucose tolerance tests were performed to ascertain if the islet reserve capacities of these Tg and WT islet recipients were comparable. The transplanted mice were then injected i.v. with multiple low doses of STZ (40 mg/kg/day×5 days) to assess the incidence of diabetes.

### Example 11

### Generation of Recombinant Proteins

Full-length cDNAs of Drak2 and p70S6 kinase were cloned into pGEX-4T-11n-frame downstream of the GST coding sequence. These constructs were named pGEX-4T-1-Drak2 and pGEX-4T-1-S6K, respectively, and were used to generate GST-tagged Drak2 and p70S6 kinase in E. coli. The recombinant proteins were purified with a size exclusion column (Superdex, 2 cm in diameter×75 cm in length,) followed by a glutathione-agarose column (GE Healthcare, Piscataway, N.J.). Drak2 cDNA was also cloned into pCEP4-HA in-frame downstream of a coding sequence of 3 HA repeats. The construct was called pCEP4-HA-Drak2 and was employed to transfect NIT-1 cells. In some experiments, HA-Drak2 was purified with Sepharose conjugated with anti-HA Ab (Covance, Berkeley, Calif.)

### Example 12

### Protein Kinase Substrate Array

Mouse recombinant Drak2 protein (95% pure according to silver staining) produced from E. coli was used as a kinase in the Protoarray Kinase Substrate Identification Kit, which contains 5000 human protein kinase substrates (Invitrogen, Carlsbad, Calif.). The reaction was conducted according to manufacturer's instructions. Proteins with a Z-score above 3 (indicating a confidence level above 99.9%) are considered potential Drak2 substrates.

Z-score=(the signal value from a given protein minus the mean signal value for all proteins in the array)/the signal value of standard deviation for all proteins.

### Example 13

### In Vitro Kinase Assay

The autophosphorylation of Drak2 were performed by incubating 0.3 μg HA-Drak2 or GST-Drak2 protein in kinase buffer (10 mM Tris-HCl [pH 7.5], 10 mM MgCl2, 3 mM MnCl2, 0.5 mM CaCl2 and 0.1 mM [-32P]-ATP (111 GBq/mmol)(GE Healthcare) in a total volume of 30 μl at 30° C. for 15 min. In some experiments, GST of GST-Drak2 and GST-p70S6 kinase was cleaved by thrombin (GE Healthcare) and then used in the in vitro kinase assay. The kinase reactions were terminated by adding 10 μl of 3×SDS-polyacrylamide gel electrophoresis (PAGE) loading buffer. The proteins were resolved by SDS-PAGE, transferred to nitrocellulose membrane, and autoradiographed.

### Example 14

### Immunoblotting

NIT-1 cells were transiently transfected with pCEP4-HA-Drak2 or empty vector pCEP4-HA. After 48 h, the cells were lysed and resolved in 10% SDS-PAGE (60 μg/lane) followed by immunoblotting. For HA-Drak2 expression, membrane was blotted with mouse anti-HA mAb (Santa Cruz, Santa Cruz, Calif.; 1:1000 dilution) followed by horse radish peroxidase (HRP)-conjugated sheep anti-mouse IgG (GE Health; 1:2000 dilution). To assess p70S6 kinase phosphorylation, the membrane was blotted with mouse anti-phospho-p70S6 kinase (Thr389) Ab (Cell Signaling, Danvers, Mass.; 1:1000 dilution) followed by HRP-conjugated sheep anti-mouse IgG (GE Health; 1:2000 dilution). The membrane was also blotted with rabbit anti-p70S6 kinase Ab (Cell Signaling, Danvers, Mass.; 1:1000 dilution) followed by HRP-conjugated donkey anti-rabbit IgG to show similar total p70S6 kinase protein. In some experiments, NIT-1 cells were stimulated with IFN-γ (1000 U/ml) plus IL-1β (0.5 ng/ml), or TNF-α (200 ng/ml) plus IL-1β (0.5 ng/ml). Twenty four hours later, they were transfected with two different Drak2 siRNAs (#592 and #1162), or with a control siRNA. After additional 24 hour, phospho-p70S6 kinase and total p70S6 kinase in the cell lysates were detected by immunoblotting as described above.

### Example 15

### Combination of Rapamycin and Drak2 siRNA

NIT-1 cells were treated with IFN-g+IL-1b for 72 hours, with or without 250 nm rapamycin. Drak2 siRNA were transfected to some cells at 24 hour, the apoptosis of cells were measured with Annexin-v staining.

Although the present invention has been described hereinabove by way of specific embodiments thereof, it can be modified, without departing from the spirit and nature of the subject invention as defined in the appended claims.

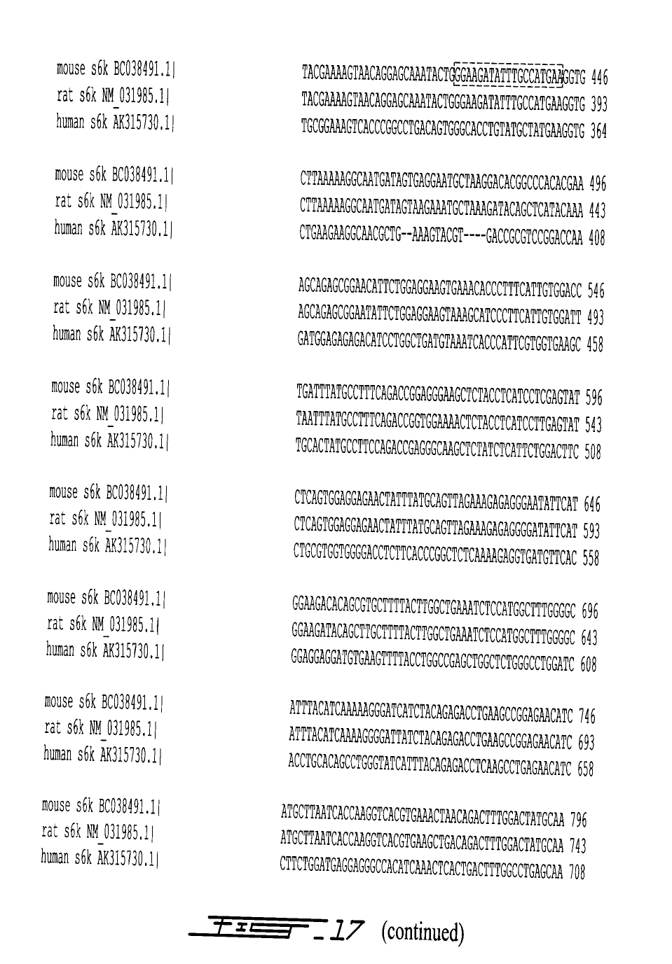

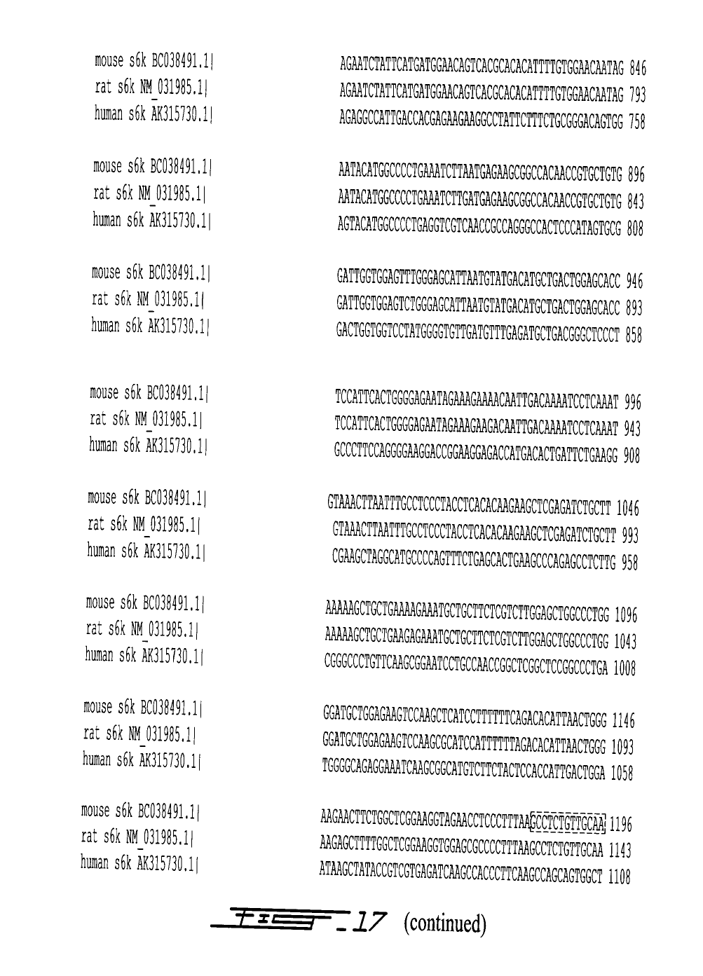

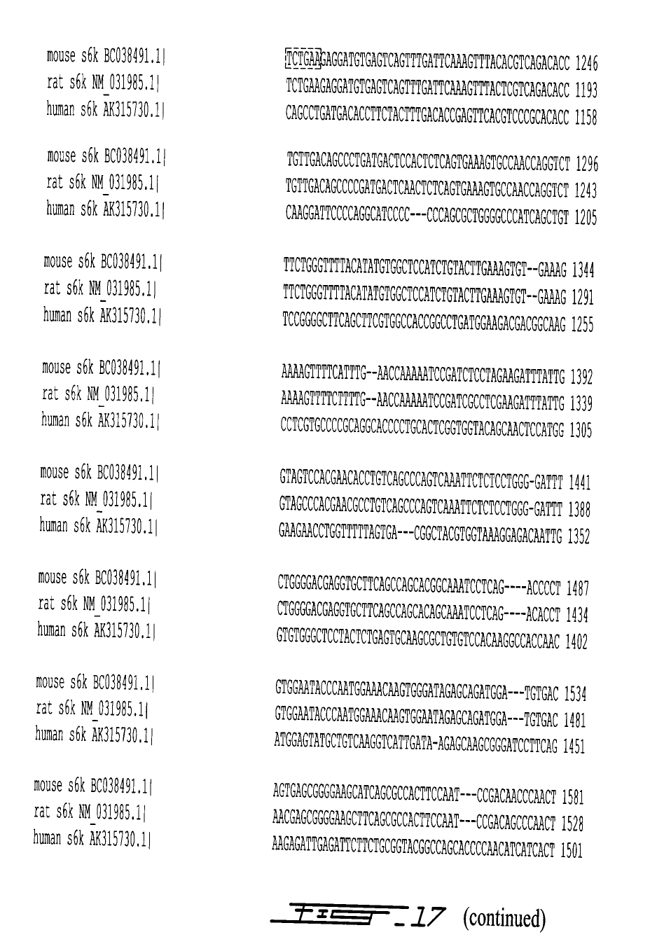

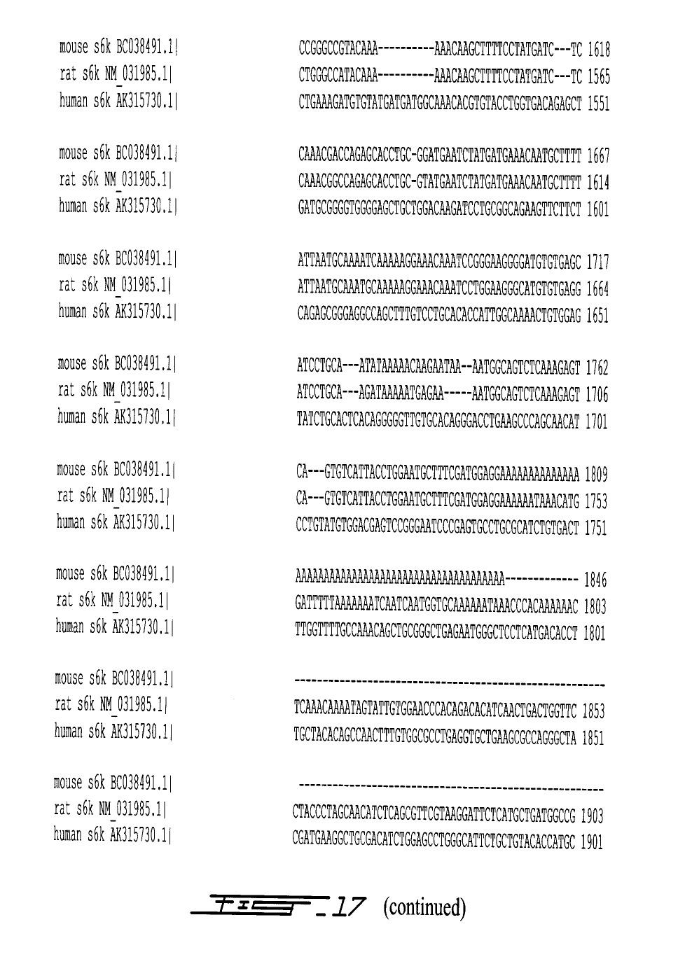

Of note, Human and mouse Drak2 protein share 85% identity and 91% homology and both belong to a family of death-associated protein kinases (DAP kinases; see FIG. 16). The role of Drak2 in human beta cell death is thus structurally implied. The conserved function has been demonstrated by the experiment using human islet cells. As shown in FIG. 19 human islets cultured in medium after 72 h presented 36.5% apoptosis. When these islets were cultured in the presence of a combination of 3 inflammatory cytokines, i.e., TNF-a, IFN-g and IL1-b, they showed increased apoptosos at the 45.7%. A combination of 2 Drak2 siRNA transfected to the islets at 24 hr after the initiation of culture reduced cytokine-induced apoptosis to 31%, while control siRNA had no effect. These data indicate that the function of mouse Drak2 in islet apoptosis, is shared by human Drak2. As islet death is a part of the pathogenesis of both type I and type 2 diabetes, it is thus concluded that human Drak2 is a T1D and T2D risk factor, the inhibition of Drak2 (alone or together with other means) will be an effective treatment to prevent or delay the onset of both T1D and T2D.

### REFERENCES

Alizadeh, B. Z., Hanifi-Moghaddam, P., Eerligh, P., van der Slik, A. R., Kolb, H., Kharagjitsingh, A. V., Pereira Arias, A. M., Ronkainen, M., Knip, M., Bonfanti, R., Bonifacio, E., Devendra, D., Wilkin, T., Giphart, M. J., Koeleman, B. P., Nolsoe, R., Mandrup, P. T., Schloot, N.C., and Roep, B. O. (2006) Clin. Exp. Immunol. 145, 480-484
Ahren, B. 2005. Type 2 diabetes, insulin secretion and beta-cell mass. Curr. Mol. Med. 5:275-286.
Chacon, M. R., Vendrell, J., Miranda, M., Ceperuelo-Mallafre, V., Megia, A., Gutierrrez, C., Fernandez-Real, J. M., Richart, C., and Garcia-Espana, A. (2007) Atherosclerosis
Cnop, M., Welsh, N., Jonas, J. C., Jorns, A., Lenzen, S., and Eizirik, D. L. (2005) Diabetes 54 Suppl 2, S97-S107
Deiss, L. P., Feinstein, E., Berissi, H., Cohen, O., and Kimchi, A. 1995. Identification of a novel serine/threonine kinase and a novel 15-kD protein as potential mediators of the gamma interferon-induced cell death. Genes Dev. 9:15-30.
Hohmeier, H. E., Tran, V. V., Chen, G., Gasa, R., and Newgard, C. B. (2003) Int. J. Obes. Relat. Metab. Disord. 27 Suppl 3, S12-S16
Inbal, B., Shani, G., Cohen, O., Kissil, J. L., and Kimchi, A. 2000. Death-associated protein kinase-related protein 1, a novel serine/threonine kinase involved in apoptosis. Mol. Cell. Biol. 20:1044-1054.
Jastrzebski, K., Hannan, K. M., Tchoubrieva, E. B., Hannan, R. D., and Pearson, R. B. (2007) Growth Factors 25, 209-226
Kahn, S. E., Hull, R. L., and Utzschneider, K. M. 2006. Mechanisms linking obesity to insulin resistance and type 2 diabetes. Nature 444:840-846.
Kawai, T., Matsumoto, M., Takeda, K., Sanjo, H., and Akira, S. 1998. ZIP kinase, a novel serine/threonine kinase which mediates apoptosis. Mol. Cell. Biol. 18:1642-1651.
Kawai, T., Nomura, F., Hoshino, K., Copeland, N. G., Gilbert, D. J., Jenkins, N. A., and Akira, S. 1999. Death-associated protein kinase 2 is a new calcium/calmodulin-dependent protein kinase that signals apoptosis through its catalytic activity. Oncogene 18:3471-3480.
Lee, M. S., Chang, I., and Kim, S. (2004) Mol. Genet. Metab 83, 82-92
Liadis, N., Murakami, K., Eweida, M., Elford, A. R., Sheu, L, Gaisano, H. Y., Hakem, R., Ohashi, P. S., and Woo, M. (2005) Mol. Cell. Biol. 25, 3620-3629
Lockwood, D. H., and Amatruda, J. M. 1983. Cellular alterations responsible for insulin resistance in obesity and type II diabetes mellitus. Am. J. Med. 75:23-31.
Mao, J., Qiao, X., Luo, H., and Wu, J. 2006. Transgenic drak2 overexpression in mice leads to increased T cell apoptosis and compromised memory T cell development. J. Biol. Chem. 281:12587-12595.
Marcelli-Tourvieille, S., Hubert, T., Moerman, E., Gmyr, V., Kerr-Conte, J., Nunes, B., Dherbomez, M., Vandewalle, B., Pattou, F., and Vantyghem, M. C. (2007) Transplantation 83, 532-538
Matsumoto, M., Miyake, Y., Nagita, M., Inoue, H., shiakubo, D., Takemoto, K., Ohtsuka, C., Murakami, H., Nakamura, N., and Kanazawa, H. 2001. A serine/threonine kinase which causes apoptosis-like cell death interacts with a calcineurin B-like protein capable of binding Na(+)/H(+) exchanger. J. Biochem. (Tokyo) 130:217-225.
Murakami, Y., Takamatsu, H., Taki, J., Tatsumi, M., Noda, A., Ichise, R., Tait, J. F., and Nishimura, S. 2004. 18F-labelled annexin V: a PET tracer for apoptosis imaging. Eur. J. Nucl. Med. Mol. Imaging. 31:469-474.
Pighin, D., Karabatas, L., Pastorale, C., Dascal, E., Carbone, C., Chicco, A., Lombardo, Y. B., and Basabe, J. C. (2005) J. Appl. Physiol 98, 1064-1069
Reynolds et al., 2004. Rational siRNA design for RNA interference. Nature Biotech. 22:326-330.
Rhodes, C. J. 2005. Type 2 Diabetes-a Matter of {beta}-Cell Life and Death? Science 307:380-384.
Sanjo, H., Kawai, T., and Akira, S. 1998. DRAKs, novel serine/threonine kinases related to death-associated protein kinase that trigger apoptosis. J. Biol. Chem. 273:29066-29071.
Turpeinen, H., Laine, A. P., Hermann, R., Simell, O., Veijola, R., Knip, M., and Ilonen, J. (2003) Eur. J. Immunogenet. 30, 289-293
Wild, S., Roglic, G., Green, A., Sicree, R., and King, H. 2004. Global prevalence of diabetes: estimates for the year 2000 and projections for 2030. Diabetes Care 27:1047-1053.
Winzell, M. S., and Ahren, B. 2004. The high-fat diet-fed mouse: a model for studying mechanisms and treatment of impaired glucose tolerance and type 2 diabetes. Diabetes 53 Suppl 3:S215-S219.
Wu, Y., Han, B., Luo, H., Roduit, R., Salcedo, T. W., Moore, P. A., Zhang, J., and Wu, J. 2003. DcR3/TR6 effectively prevents islet primary nonfunction after transplantation. Diabetes 52:2279-2286.
Wu, Y., Han, B., Luo, H., Shi, G., and Wu, J. 2004. Dipeptide boronic acid, a novel proteasome inhibitor, prevents islet-allograft rejection. Transplantation 78:360-366.

1. A method for preventing or delaying the onset of Type 1 or Type 2 diabetes in a subject, which comprises an inhibition of the level and/or activity of Drak2 in said subject's tissue or cells.

2. The method of claim 1, wherein islet cells are targeted.

3. The method of claim 1, wherein, said delaying or preventing is carried-out using an inhibitor of Drak2 level or activity.

4. The method of claim 3, wherein said inhibitor is a nucleic acid, a propetin, a peptide, a ligand or a small molecule.

5. A composition for preventing or reducing Type 1 or Type 2 diabetes in a patient comprising an inhibitor of Drak2 level or function, together with a pharmaceutically acceptable carrier.

6. The method of claim 1, further comprising a use of an inhibitor of p70S6 kinase.

7. The method of claim 6, further comprising a use of an inhibitor of cytokine function involved in diabetes onset or development.

8. A composition for preventing or reducing Type 1 or Type 2 diabetes in a patient comprising an inhibitor of Drak2 level or function, together with a pharmaceutically acceptable carrier.

9. The composition of claim 8, wherein said inhibitor is an siRNA which targets Drak2.

10. The composition of claim 9, further comprising an inhibitor of p70s6 kinase function or level.

11. A method for diagnosing a risk of developing Type 1 or Type 2 diabetes in a susceptible subject, which comprises the step of measuring a level of Drak2 activity in said susceptible subject's tissue or cells, wherein a measuring of a higher level thereof in said susceptible subject as compared to that in a control subject indicates a risk of developing diabetes in said susceptible subject.
